(19) United States
(12) Patent Application Publication
(10) Pub. No.: US 2005/0148034 A1
(43) Pub. Date: Jul. 7, 2005

(54) Methods for identification of modulators of angiogenesis, compounds discovered thereby, and methods of treatment using the compounds

(76) Inventors: Robert J. Hariri, Florham Park, NJ (US); Faribourz Payvandi, Belle Mead, NJ (US); Lei Wu, Bridgewater, NJ (US); David I. Stirling, Branchburg, NJ (US); Qian Ye, Livingston, NJ (US)

Correspondence Address: JONES DAY, 222 EAST 41ST ST, NEW YORK, NY, 10017, US

(21) Appl. No.: 10/511,354

(22) PCT Filed: Apr. 14, 2003
(86) PCT No.: PCT/US03/11578

Related U.S. Application Data
(60) Provisional application No. 60/372,127, filed on Apr. 12, 2002.

(51) Int. Cl. G01N 33/574; C12N 5/08

(52) U.S. Cl. 435/7.23; 435/366

(57) ABSTRACT

The present invention relates to methods of identifying modulators of angiogenesis utilizing human cells. The methods of the invention can be employed to assay compounds and small molecules for their ability to modulate human angiogenesis utilizing human pluripotent stem cells in an in vitro assay system. The present invention further relates to methods of identifying modulators of human angiogenesis by determining the ability of a test compound to modulate spontaneous vasogenesis in an in vitro assay system utilizing nonembryonic pluripotent stem cells. The present invention relates to in vitro assay systems utilizing nonembryonic pluripotent stem cells for the identification of compounds that modulate human angiogenesis or human vasogenesis. The present invention also relates to methods of treatment which require modulation of human angiogenesis or vasogenesis comprising administering to patients in need of such treatment compounds or small molecules which have been identified to be inhibitors of human angiogenesis or vasogenesis.

This application claims benefit of U.S. Provisional Application No. 60/372,127, filed Apr. 12, 2002, which is incorporated herein by reference in its entirety.

### 1. INTRODUCTION

The present invention relates to methods of identifying modulators of angiogenesis utilizing vessel cells or nonembryonic stem cells. The methods of the invention can be employed to assay compounds and small molecules for their ability to modulate human angiogenesis utilizing human pluripotent stem cells in an in vitro assay system. The present invention further relates to methods of identifying modulators of human angiogenesis by determining the ability of a test compound to modulate spontaneous vasogenesis in an in vitro assay system utilizing nonembryonic pluripotent stem cells. The present invention relates to in vitro assay systems utilizing nonembryonic pluripotent stem cells for the identification of compounds that modulate human angiogenesis or human vasogenesis. The present invention also relates to methods of treatment that require modulation of human angiogenesis or vasogenesis comprising administering to patients in need of such treatment compounds or small molecules which have been identified to be inhibitors of human angiogenesis or vasogenesis.

### 2. BACKGROUND OF THE INVENTION

There is considerable interest in the identification and generation of compounds that modulate human angiogenesis. The major obstacle in identifying compounds which modulate human angiogenesis and vasogenesis is the lack of in vitro assay systems which truly mimic human angiogenesis and vasogenesis as these processes occur in vivo.

Several disease processes have been demonstrated to require the invasion or migration of endothelial cells as part of their pathology, including tumor invasion, tumor metastasis, pathological angiogenesis, inflammation and endometriosis (Aznavoorian et al., 1993, Cancer 71(4): 1368-1383; Fernandez et al., 1995, Fertil. and Steril. 63(1): 45-51; Fox et al., 1996, J. Pathol. 179: 232-237; Lennarz et al., 1991, Biochim. Biophys. Acta 1071: 149-158; Liotta et al., 1991, Cell 64: 327-336; Mareel et al., 1990, Cancer and Metastasis Rev. 9: 45-62; and Osborn 1990, Cell 62: 3-6.). Angiogenesis is also involved in many other diseases and conditions which are angiogenesis-dependent, including arthritis and atherosclerotic plaques, diabetic retinopathy, neovascular glaucoma, trachoma and corneal graft neovascularization, psoriasis, scleroderma, hemangioma and hypertrophic scarring, vascular adhesions and angiofibroma.

Angiogenesis is the process of new blood vessel formation from pre-existing vessels. Vasogenesis is the process of tube formation from a monolayer of endothelial cells. Under normal physiological conditions, humans or animals undergo angiogenesis and vasogenesis in very specific situations, such as wound healing, fetal and embryonal development and the formation of the corpus luteum, endometrium and placenta.

Endothelial cells form a single layer of cells that lines all blood vessels and regulates exchanges between the blood stream and surrounding tissues. New blood vessels develop from the walls of existing small vessels by the outgrowth of these endothelial cells, which have the capacity to form hollow capillary tubes even when isolated in culture. Once the vascular system is fully developed, endothelial cells of blood vessels normally remain quiescent with no new vessel formation. If disease or injury occurs, the formation of new blood vessels can proceed normally, as in natural wound healing. Insufficient formation of new blood vessels may result in chronic dermal ulcers. Alternatively, a deregulation of growth can give rise to an abnormal increase in vessel density as in tumorigenesis, diabetic retinopathy, psoriasis and inflammation. Thus, inhibition of inappropriate angiogenesis or enhancement of angiogenesis in non-healing wounds is therefore an extremely important target for drug discovery programs. However research in this area has been hindered by the lack of in vitro models of angiogenesis that accurately mimic the vessels' natural environment in vivo.

Angiogenesis is an extremely complex process which involving a wide range of growth factors, extracellular matrix molecules, enzymes and various cell types. Such a complexity of relationships has resulted in major difficulties in developing an in vitro assay which models the entire in vivo process. Angiogenesis can be subdivided into three phases: proliferation, migration and differentiation. Assays exist which model each of these phases separately. In particular, simple in vitro assays measure changes in proliferation of a range of cell types and assess migration over basement membrane proteins. Current in vitro assay systems, which depend on provision of a protein matrix, generally measure the ability of endothelial cells to form vessels. Assay systems measuring differentiation involve formation of cord-like structures by endothelial cells. All such systems depend on supplying the cells with exogenous basement proteins on which the cells migrate to form tubules. However, the problem with these assays is that none of them combine all of the stages required for angiogenesis.

One in vitro model system is the rat aortic ring model. In the rat aorta ring model, rat aorta ring explant cultures are utilized under short term and long term maintenance conditions. In this assay system, rat aorta ring segments are cultured under short term maintenance conditions for three to four days in order to obtain pure populations of endothelial and muscle cells. By contrast, long term rat aorta ring explant cultures allow for the coordinated outgrowth and proliferation of both endothelial and smooth muscle cells (Diglio et al., 1989, Laboratory Investigation 60(4): 523-531).

Recently, another group has attempted to generate a human in vitro assay for studying angiogenesis, and in doing so have utilized embryonic aortic ring explants from 11 to 12 day old embryos embedded in collagen gels (Allesandri et al., 2001, Laboratory Investigation 81(6): 875-885).

Other in vitro assays that model the combined stages of angiogenesis include the use of blood vessel fragment from human placental tissues obtained within 6 hours of birth (Parish et al., U.S. Pat. No. 5,976,782), the use of commercially available porcine carotid arteries (Stiffey-Wilusz, U.S. Patent Application No. 2001/0046666), and the use of a dual culture of endothelial cells and fibroblasts (Grant et al., WO 99/17116; Grant et al., U.S. Patent Application No. 2001/0005581). By seeding the dual culture with a cell ratio of about 2:1 to 8:1 of human adult dermal fibroblasts to human umbilical vein endothelial cells, the multicellular model most closely resembles in vivo angiogenesis (Grant et al., WO 99/17116; Grant et al., U.S. Patent Application No. 2001/0005581).

To date, however, no angiogenesis model utilizes stem cells, or stem cells in combination with vessel tissue, or tumor cells in combination with either stem cells or sections of vessel tissue. It is believed that angiogenesis assays utilizing these cells will more accurately reflect the angiogenesis process than previously-described assays.

### 3. SUMMARY OF THE INVENTION

The present invention relates to in vitro assay systems utilizing human pluripotent stem cells for the identification of compounds which modulate human angiogenesis or human vasogenesis. In a preferred embodiment, the human pluripotent stem cells are placental in origin. The screening assays of the present invention can be used to identify compounds which inhibit or stimulate angiogenesis and/or vasogenesis.

The present invention relates to assays to screen for modulators of angiogenesis comprising culturing human pluripotent stem cells with portions of blood vessels, i.e., vessel rings, under conditions to allow for angiogenesis and determining the effect that test compounds have on the angiogenesis process. In a preferred embodiment of the invention, the pluripotent stem cells are nonembryonic in origin. In a preferred embodiment of the invention, the nonembryonic stem cells are placental derived stem cells. In another preferred embodiment of the invention, the portions of blood vessels are human in origin, preferably human umbilical cord.

The invention also preferably provides assays to screen for modulators of angiogenesis comprising culturing vessel rings, or stem cells, in the presence of tumor cells, under conditions to allow for angiogenesis, and determining the effect that test compounds have on the angiogenesis process.

Preferably the screening assay of the invention comprises the steps of: (a) providing in a suitable growth container a culture medium suitable for sustaining at least growth of endothelial cells; (b) culturing for at least 24 hours in said growth container a sample of human vessel, said vessel being free of connective tissue; (c) changing the culture medium at regular intervals; and (d) monitoring the formation of microvessel outgrowth.

Thus, in one embodiment, the invention provides a method of identifying a modulator of angiogensis comprising: (a) culturing a plurality of stem cells in the presence of a test compound, for a time and under conditions suitable for the growth of endothelial cells; and (b) comparing the amount of microvessel outgrowth from said stem cells in the presence of said test compound as compared to a control amount of vessel outgrowth, wherein if said microvessel outgrowth is greater or less than said control level of microvessel outgrowth, the test compound is identified as a modulator of angiogenesis. In a specific embodiment, said stem cells are cultured with a vessel section. In another specific embodiment, said stem cells are cultured with a plurality of tumor cells. In more specific embodiment, said tumor cells are cells of a tumor cell line. In another specific embodiment, said stem cells are additionally cultured in the presence of hydrocortisone, epidermal growth factor, or bovine brain extract. In yet another specific embodiment, said modulator of angiogenesis is identified as an anti-angiogenic agent. In another specific embodiment, said modulator of angiogenesis is identified as an angiogenic agent. In another specific embodiment, said culturing of a plurality of stem cells in the presence of a test compound is for at least seven days. In another specific embodiment, said culturing of a plurality of stem cells in the presence of a test compound is for at least fourteen days. In yet another specific embodiment, said stem cells are cultured on a matrix that comprises fibrin. In another specific embodiment, said stem cells are cultured in a physiological gel that comprises fibrin. In another specific embodiment, said stem cells are cultured in a physiological gel that comprises non-denatured collagen.

In another embodiment, the invention provides a method of identifying a modulator of angiogensis comprising: (a) culturing a vessel section in the presence of a plurality of tumor cells and a test compound, for a time and under conditions suitable for the growth of endothelial cells and said tumor cells; and (b) comparing the amount of microvessel outgrowth from said vessel section in the presence of said test compound as compared to a control amount of microvessel outgrowth, wherein if said microvessel outgrowth is greater or less than said control level of microvessel outgrowth, the test compound is identified as a modulator of angiogenesis.

The present invention also provides methods of treating individuals with compounds identified in the above assay. In this aspect, the present invention relates to methods of treatment that require modulation of human angiogenesis or vasogenesis comprising administering to patients in need of such treatment compounds or small molecules which have been identified to be inhibitors of human angiogenesis or vasogenesis. The present invention also relates to methods of treatment which require modulation of human angiogenesis or vasogenesis comprising administering to patients in need of such treatment compounds or small molecules which have been identified to be stimulators of human angiogenesis or vasogenesis.

Thus, in one embodiment, the invention provides a method of treating an individual, said individual having a disease or condition that is associated with abnormal vessel growth, comprising administering to said individual a therapeutically effective amount of a TNF-α inhibitor. In a specific embodiment, said TNF-α inhibitor is an IMiD™. In another specific embodiment, said IMiD™ is Actimid™ or Revimid™. In another specific embodiment, said disease or condition is cancer. In more specific embodiment, said cancer is a metastatic cancer. In another more specific embodiment, said cancer is breast cancer. In another specific embodiment, said disease or condition is selected from the group consisting of inflammation, endometriosis, arthritis, atherosclerotic plaques, diabetic retinopathy, neovascular glaucoma, trachoma, corneal graft neovascularization, psoriasis, scleroderma, hemangioma and hypertrophic scarring, vascular adhesions and angiofibroma.

The invention also provides methods of inhibiting angiogenesis in any context. Thus, the invention provides a method of inhibiting angiogenesis, comprising contacting a plurality of cells, said plurality of cells being capable of forming a vessel, with an inhibitor of TNF-α. In a specific embodiment, said inhibitor of TNF-α is Actimid™ or Revimid™. In another specific embodiment, said plurality of cells is a plurality of cells within an individual. In another specific embodiment, said plurality of cells is a plurality of cells in cell culture.

The present invention also relates to angiogenesis assay kits comprising a sample of placental derived stem cells and a sample of human umbilical cord. In another embodiment of the invention, the assay kits further comprise a sample of human cord blood plasma.

Examples of test compounds which may be used in connection with the screening assays of the invention include, but are not limited to small molecules, organic compounds, inorganic compounds, polypeptides, peptides, proteins, hormones, cytokines, oligonucleotides, nucleic acids or other macromolecules. Other examples of the small molecule compounds that may be used in connection with the invention, include, but are not limited to, compounds that inhibit TNF-α activity. Preferably, the molecular weight of the compound is less than 1000 grams/mole. Such compounds include, but are not limited to, cyano and carboxy derivatives of substituted styrenes, the cyclic imides, the cycloalkyl amides and cycloalkyl nitrites, the aryl amides, the 1-oxo-2-(2,6-dioxo-3-fluoropiperidin-3yl)isoindolines and 1,3-dioxo-2-(2,6-dioxo-3-fluoropiperidine-3-yl)isoindolines, the tetra substituted 2-(2,6-dioxopiperdin-3-yl)-1-oxoisoindolines, the imide/amide ethers and alcohols, the succinimides and maleimides, 1-Oxo and 1,3 dioxo-2-(2,6-dioxopiperidin-3 yl)isoindolines, non-polypeptide cyclic amides, imido and amido substituted alkanohydroxamic acids, substituted phenethylsulfones, thalidomide, aminothalidomide, 3-(4-Amino-1-oxo-1,3-dihydro-isoindol-2-yl)-piperidine-2,6-dione, as well as analogs, hydrolysis products, metabolites, derivatives and precursors of thalidomide, aminothalidomide, and 3-(4-Amino-1-oxo-1,3-dihydro-isoindol-2-yl)-piperidine-2,6-dione, aryl amides, substituted 2-(2,6-dioxopiperidin-3-yl) phthalimies and substituted 2-(2,6-dioxopiperidin-3-yl)-1-oxoisoindoles, and isoindole-imide compounds. In one embodiment, the preferred compounds are thalidomide, as well as analogs, hydrolysis products, metabolites, derivatives and precursors of thalidomide.

In another embodiment, the compounds are IMiDS™, including but not limited to Actimid™, and Revimid™ (Celgene Corp., Warren, N.J.), or SeICIDs™.

In another embodiment of the invention, the stem or progenitor cells are derived not from a postpartum perfused placenta but instead, are isolated from other sources such as cord blood, bone marrow, peripheral blood or adult blood.

3.1 Definitions

As used herein, the terms “angiogenesis” and “vasogenesis” refer to the generation of new blood vessels.

As used herein, the term “bioreactor” refers to an ex vivo system for propagating cells, producing or expressing biological materials and growing or culturing cells tissues, organoids, viruses, proteins, polynucleotides and microorganisms.

As used herein, the term “embryonic stem cell” refers to a cell that is derived from the inner cell mass of a blastocyst (e.g., a 4- to 5-day-old human embryo) and that is pluripotent.

As used herein, the term “embryonic-like stem cell” refers to a cell that is not derived from the inner cell mass of a blastocyst. As used herein, an “embryonic-like stem cell” may also be referred to as a “placental stem cell.” An embryonic-like stem cell is preferably pluripotent. However, the stem cells, which may be obtained from the placenta, include embryonic-like stem cells, multipotent cells, and committed progenitor cells. According to the methods of the invention, embryonic-like stem cells derived from the placenta may be collected from the isolated placenta once it has been exsanguinated and perfused for a period of time sufficient to remove residual cells.

As used herein, the term “endothelium” refers to a thin layer of flat epithelial cells that normally line serous cavities, lymph vessels, and blood vessels.

As used herein, the term “exsanguinated” or “exsanguination,” when used with respect to the placenta, refers to the removal and/or draining of substantially all cord blood from the placenta. In accordance with the present invention, exsanguination of the placenta can be achieved by, for example, but not by way of limitation, draining, gravity induced efflux, massaging, squeezing, pumping, etc. In a preferred embodiment, exsanguination of the placenta may further be achieved by perfusing, rinsing or flushing the placenta with a fluid that may or may not contain agents, such as anticoagulants, to aid in the exsanguination of the placenta.

As used herein, the term “perfuse” or “perfusion” refers to the act of pouring or passaging a fluid over or through an organ or tissue, preferably the passage of fluid through an organ or tissue with sufficient force or pressure to remove any residual cells, e.g., non-attached cells from the organ or tissue. As used herein, the term “perfusate” refers to the fluid collected following its passage through an organ or tissue. In a preferred embodiment, the perfusate contains one or more anticoagulants.

As used herein, the term “endogenous cell” refers to a “non-foreign” cell, i.e., a “self” or autologous cell, that is derived from the placenta.

As used herein, the term “exogenous cell” refers to a “foreign” cell, i.e., a heterologous cell (i.e., a “non-self” cell derived from a source other than the placental donor) or autologous cell (i.e., a “self” cell derived from the placental donor) that is-derived from an organ or tissue other than the placenta.

As used herein, the term “organoid” refers to an aggregation of one or more cell types assembled in superficial appearance or in actual structure as any organ or gland of a mammalian body, preferably the human body.

As used herein, the term “multipotent cell” refers to a cell that has the capacity to grow into any of subset of the mammalian body's approximately 260 cell types. Unlike a pluripotent cell, a multipotent cell does not have the capacity to form all of the cell types.

As used herein, the term “pluripotent cell” refers to a cell that has complete differentiation versatility, i.e., the capacity to grow into any of the mammalian body's approximately 260 cell types. A pluripotent cell can be self-renewing, and can remain dormant or quiescent within a tissue. Unlike a totipotent cell (e.g., a fertilized, diploid egg cell), an embryonic stem cell cannot usually form a new blastocyst.

As used herein, the term “progenitor cell” refers to a cell that is committed to differentiate into a specific type of cell or to form a specific type of tissue.

As used herein, the term “stem cell” refers to a master cell that can reproduce indefinitely to form the specialized cells of tissues and organs. A stem cell is a developmentally pluripotent or multipotent cell. A stem cell can divide to produce two daughter stem cells, or one daughter stem cell and one progenitor (“transit”) cell, which then proliferates into the tissue's mature, fully formed cells.

As used herein, the term “totipotent cell” refers to a cell that is able to form a complete embryo (e.g., a blastocyst).

As used herein, the term “vasogenesis” refers to generation or formation of tubes or microtubules.

As used herein, the term “vessel ring” means a section of vessel. Generally the vessel section is a cross-section that appears to be ring-shaped, but may be any section of vessel that is culturable. The vessel may be any vessel (i.e., arterial, venous, lymphatic, etc.)

### 4. BRIEF DESCRIPTION OF THE FIGURES

FIGS. 1(A-D). Photomicrographs of cultured cells in umbilical vessel ring assays as described in Section 6.2. A. Positive control. The explant was cultured in media+EGCF 200 μg/ml. Numerous cells that migrated from the explant surround the explant and the individual cells exhibited extensive outgrowth. B. Negative control. The explant was cultured in placental conditioned media+supplement. In the absence of EGCF, fewer cells migrated from the explant than in the positive control (A). C. Treatment Group 3. The explant was cultured in placental conditioned media+EGCF 200 μg/ml+Thalomid™ 100 μg/ml. In the presence of 100 μg/ml of Thalomid™, cells migrated a shorter distance from the explant than in the positive control (A). D. Treatment Group 2. The explant was cultured in placental conditioned media+EGCF 200 μg/ml+Thalomid™ 10 μg/ml. In the presence of 10 μg/ml of Thalomid™, cells migrated a shorter distance from the explant and they exhibited less dense outgrowth than in the positive control (A).

FIGS. 2(A-C). Photomicrographs of cultured cells in umbilical vessel ring assays as described in Section 6.2. A. Control. Cells were cultured in placental conditioned media+ECGF 200 μg/ml+DMSO 1 μg/ml. B. Cells were cultured in placental conditioned media+ECGF 200 μg/ml+DMSO 1 μg/ml+Thalomid™ 1 μg/ml. Fewer cells are seen than in the control (A). B. Cells were cultured in placental conditioned media+ECGF 200 μg/ml+DMSO 1 μg/ml+Thalomid™ 10 μg/ml. Fewer cells are seen than in the control (A) or in (B).

FIGS. 3(A-B). Photomicrographs of cultured cells in umbilical vessel ring assays as described in Section 6. A. Control. Cells were cultured in placental conditioned media+DMSO. Cells exhibit predominantly a non-branching (e.g., endothelial) phenotype. B. Cells were cultured in placental conditioned media+DMSO+Thalomid™. More cells exhibit a branching (e.g., neuronal) phenotype than in the control (A).

FIG. 4. Graphic representation of the effects of different concentrations of Thal1, Actimid™ (CC-4047), and Fumagillin on human angiogenesis.

FIG. 5. Pictomicropgraphs of placental embryonic-like stem cells cultured in an umbilical vessel ring assay as described in Section 6.3 in the presence of varying concentrations of Thall, Actimid™ (CC-4047) and Fumagillin.

FIG. 6. Graphic depiction of umbilical vessel ring assay.

### 5. DETAILED DESCRIPTION OF THE INVENTION

The present invention relates to in vitro assay systems utilizing human pluripotent stem cells for the identification of compounds that modulate human angiogenesis or human vasogenesis. The screening assays of the present invention can be used to identify compounds that inhibit or stimulate angiogenesis and/or vasogenesis.

The present invention relates to assays to screen for modulators of angiogenesis comprising culturing human pluripotent stem cells or portions of blood vessels under conditions to allow for angiogenesis and determining the effect that test compounds have on the angiogenesis process. In a preferred embodiment of the invention, the pluripotent stem cells are nonembryonic in origin. In a particularly preferred embodiment of the invention, the nonembryonic stem cells are placental derived stem cells. In another preferred embodiment of the invention, the portions of blood vessels are human in origin, and are preferably derived from human umbilical cord. In another embodiment of the invention, the stem or progenitor cells are derived not from a postpartum perfused placenta, but are isolated from other sources such as cord blood, bone marrow, peripheral blood or adult blood.

The present invention encompasses in vitro screening assays for identifying modulators of angiogenesis, which assays rely on the co-culture of human pluripotent stem cells with vessels derived from human umbilical cord. In a preferred embodiment, the human pluripotent stem cells are placental in origin.

The present invention also relates to angiogenesis assay kits comprising a sample of placental derived stem cells and a sample of human umbilical cord. In another embodiment of the invention, the assay kits further comprise a sample of human cord blood plasma.

The present invention also relates to methods of treatment that require modulation of human angiogenesis or vasogenesis comprising administering to patients in need of such treatment compounds or small molecules which have been identified to be inhibitors of human angiogenesis or vasogenesis. The present invention also relates to methods of treatment that require modulation of human angiogenesis or vasogenesis, comprising administering to patients in need of such treatment compounds or small molecules that have been identified to be stimulators of human angiogenesis or vasogenesis.

Examples of test compounds which may be used in connection with the screening assays of the invention include, but are not limited to small molecules, organic compounds, inorganic compounds, polypeptides, peptides, proteins, hormones, cytokines, oligonucleotides, nucleic acids or other macromolecules.

Examples of small molecule compounds that may be used in the treatment methods described herein include, but are not limited to, compounds that inhibit TNF-α activity. Such compounds include, but are not limited to, cyano and carboxy derivatives of substituted styrenes, the cyclic imides, the cycloalkyl amides and cycloalkyl nitrites, the aryl amides, the 1-oxo-2-(2,6-dioxo-3-fluoropiperidin-3yl)isoindolines and 1,3-dioxo-2-(2,6-dioxo-3-fluoroipiperidine-3-yl) isoindolines, the tetra substituted 2-(2,6-dioxopiperdin-3-yl)-1-oxoisoindolines, the imide/amide ethers and alcohols, the succinimides and maleimides, 1-Oxo and 1,3 dioxo-2-(2,6-dioxopiperidin-3 yl)isoindolines, non-polypeptide cyclic amides, imido and amido substituted alkanohydroxamic acids, substituted phenethylsulfones, thalidomide, aminothalidomide, 3-(4-Amino-1-oxo-1,3-dihydro-isoindol-2-yl)-piperidine-2,6-dione, as well as analogs, hydrolysis products, metabolites, derivatives and precursors of thalidomide, aminothalidomide, and 3-(4-Amino-1-oxo-1,3-dihydro-isoindol-2-yl)-piperidine-2,6-dione, aryl amides, substituted 2-(2,6-oxopiperidin-3-yl) phthalimies and substituted 2-(2,6-dioxopiperidin-3-yl)-1-oxoisoindoles, and isoindole-imide compounds. In one embodiment, the preferred compounds are thalidomide, as well as analogs, hydrolysis products, metabolites, derivatives and precursors of thalidomide.

Any human stem cell can be used in accordance with the methods of the invention, including but not limited to, stem cells isolated from cord blood (“CB” cells), placenta and other sources. The stem cells may include pluripotent cells, i.e., cells that have complete differentiation versatility, that are self-renewing, and can remain dormant or quiescent within tissue. The stem cells may also include multipotent cells or committed progenitor cells. In one preferred embodiment, the invention utilizes stem cells that are viable, quiescent, pluripotent stem cells that exist within the full-term placenta can be recovered following successful birth and placental expulsion, exsanguination and perfusion resulting in the recovery of multipotent and pluripotent stem cells.

5.1 Screening Assays to Identify Modulators of Angiogenesis

The present invention encompasses screening assays to identify modulators of angiogenesis comprising screening for the ability of a test compound to modulate vasogenesis or tube formation. In accordance with this aspect of the invention, human pluripotent stem cells or vessel rings are gown in culture and contacted with test compounds, and the effect on angiogenesis is determined.

5.1.1 Assay Methods

The present invention provides a method for identifying modulators of vasogenesis or angiogenesis, wherein vessels arise from plated stem cells. Stem cells are plated, and adherent cells are separated from non-adherent populations, preferably after 24 hours of culture. Adherent cells are cultivated in suitable culture medium. Any suitable culture medium is encompassed within the method; a preferred medium is DMEM supplemented with 5-20% cord blood serum (CBS) and antibiotics. Preferably, the medium is further supplemented with hydrocortisone, epidermal growth factor and/or bovine brain extract. Culture of the stem cells results in spontaneous vasogenesis. Spontaneous vasogenesis may be characterized by the assembly of microtubular structures. In this method, test compounds are assayed for their ability to modulate the assembly of these microtubule structures. Inhibitors of angiogenesis may be identified on the basis of their ability to prevent or decrease the process of microtubule formation as compared to a control, for example, assay conditions in the absence of test compound. Conversely, stimulators of angiogenesis may be identified on the basis of their ability to enhance or increase the process of microtubule formation as compared to a control, for example, assay conditions in the absence of the test compound.

In one embodiment, the present invention provides a method for screening substances for angiogenesis modulation activity comprising culturing nonembryonic pluripotent stem cells from a biological sample together with a physiological gel, suitable nutrients and at least one substance suspected of having angiogenesis modulation activity for a time and under conditions sufficient to allow growth of new vascular tissue, examining said fragment for new vascular tissue growth and comparing said growth to that of a control. The term “angiogenesis modulation” refers to the ability of a substance to modulate or change normal angiogenic activity of the blood vessel fragments and includes inhibition, promotion, and enhancement of angiogenic activity. The method may be used to test compounds or substances which are possible angiogenesis inhibitors, promoters, or enhancers. The term “biological sample” refers to any sample that is ultimately derived from an animal tissue where it is desirable to test whether a substance has angiogenesis modulation activity for that particular tissue and/or animal species. Preferably the biological sample is derived from human tissue.

Stem cells that may be used in accordance with the invention include, but are not limited to, cord blood (CB) stem cells, placental stem cells, embryonic stem (ES) cells, embryonic-like stem cells, trophoblast stem cells, progenitor cells, and multipotent, pluripotent and totipotent cells. In a preferred embodiment, nonembryonic pluripotent stem cells are used for both the control and the cultures being screened with test compounds having potential angiogenesis modulation activity.

The present invention also encompasses identifying modulators of vasogenesis or angiogenesis, wherein vessels arise from cultured vessel rings, i.e., sections of vessel grown in vitro. In accordance with this aspect of the invention, sections of vessel rings, preferably obtained from umbilical cord, are cultured under conditions to allow for vessel outgrowth. In one embodiment, blood vessels approximately 1-2 mm in diameter and 1-2 cm in length are excised from human umbilical cord. Preferably, such excision is performed within 12 to 24 hours of birth. Both arterial and venous tissue are harvested and maintained separately. The vessels are placed in culture medium, such as DMEM containing 2.5 μg/ml of fungizone, and cut into 1-2 mm length sections. Vessel fragments are preferably freed of residual clots and soaked in culture medium before use. Dissecting and sectioning of vessels is best performed with the aid of a surgical microscope. Blood vessels of venular or arterial origin may also be used. Preferably, for each experiment, vessel fragments from only one vessel are be used.

The vessel outgrowth assays are performed in petri dishes or multi-well culture plates (Costar, Cambridge, Mass.). The culture dishes are preferably prepared by pre-coating with either 0.1% gelatin (Sigma, St. Louis, Mo.) or Matrigel to form a matrix. Following coating, the culture dishes are coated with culture medium. As an exemplary embodiment of the invention, following coating of plates, 50 μl of human cord blood plasma in 5 mL of DMEM is added to each dish/well to form a surface film over the matrix. The film is allowed to set at 37° C. for 90 minutes after which it is removed leaving a thin film in each dish/well. Once preparation of the culture dishes is complete, vessel ring segments are placed in the culture dishes.

Vessel ring segments generally adhere to the matrix materials within 12 hours, allowing the addition of medium without detachment of the vessel segments due to buoyancy. Following adherence, vessels are cultured at 37° C. in a humidified environment for 7-21 days. Preferably, the medium is changed at regular intervals, e.g., 72 hour intervals. Exemplary culture conditions comprise maintaining the cultures in DMEM supplemented with 20% human cord blood plasma, L-glutamine, penicillin/streptomycin and heparin. Preferably, the medium is further supplemented with hydrocortisone, epidermal growth factor and/or bovine brain extract. In a preferred embodiment, the blood vessel fragment is cultured for a time sufficient to establish a good angiogenic response prior to the substance being administered, such as, for example, 14 days prior to administration. The extent of this response is then preferably quantified and recorded.

Test compounds are administered during culture to determine any modulation of angiogenesis. The test compound may be administered at a change of medium, or may be added separately at any time during culture. Preferably, test compounds are added once the stem cells or vessel rings are adherent, and culture continues for the full 7-21 days. However, test compounds may be added at other times. For example, vessel outgrowth may be allowed in medium for 1, 2, 3, 4, 5 6, 7, 8, 9, 10 or more days, followed by a single administration of the test compound. Each test compound will be evaluated at various concentrations to enable generation of a dose-response analysis. Positive control may be defined as, for example, the response (e.g., microvessel outgrowth) to endothelial cell growth supplement (ECGS; 200 μg/ml; Collaborative Research, Bedford, Mass.) and negative control may be defined, for example, as the response to media alone. Vessel outgrowth may be scored both as quantitative comparison to positive and negative controls as defined in table below, and morphometrically as both maximal distance of vessel sprout growth in microns from the vessel ring and as the total area of endothelial cell coverage (ECA)/area of vessel ring (VRA).

In yet another embodiment of the screening assays of the invention, a small section of human umbilical vessel rings obtained from umbilical arteries is embedded in a solution, such as MATRIGEL® plus human collagen, and cultured in an optimized medium, preferably serum free medium containing growth factors. The umbilical vessel rings may be cultured for one to four weeks, optimally three weeks, or until such time that microvessels develop from the rings. Test compounds can be assayed for their ability to inhibit or enhance the growth of microvessels as an indication of their ability to inhibit or enhance angiogenesis.

In a combination of the above two methods, vessel rings are obtained and plated as above, and are cultured in the presence of stem cells, also obtained as above. The vessel rings and stem cells are co-cultured for 7-21 days, at which time the extent of vessel outgrowth is determined. Here, any culture medium that allows the growth of endothelial cells, and other cells, may be used. It is expected that the addition of stem cells will result in the differentiation of these cells into cell types that will facilitate the development of vessels, thus re-creating the vessels' natural environment more closely than other assay methods. As above, test and/or control compounds may be added to the culture medium at the start of culture, or at any time during culture.

Thus, in one embodiment, the present invention provides a method for determining the ability of a substance to modulate (i.e., either prevent or stimulate) growth of new vascular tissue and/or induce regression of new vascular tissue comprising culturing nonembryonic pluripotent stem cells together with a vessel section, physiological gel and suitable nutrients for a time sufficient to allow growth of new vascular tissue, administering the substance to said fragment, and culturing said fragment together with suitable nutrients for a time, then examining said fragment to determine whether prevention of new vascular tissue growth and/or regression of new vascular tissue has occurred.

In another embodiment, said stem cells or vessel rings may be co-cultured with tumor cells, particularly cells having an origin in metastatic cancer. Because many metastatic or aggressive cancers have an angiogenic component (that is, the tumor secretes factors that encourage angiogenesis), such a co-culture will recreate the natural environment of a tumor. Tumor cells used in such a co-culture may be tumor cells obtained directly from an individual, cells obtained from an individual and stored, or any of a number of immortalized tumor cell lines know to those of skill in the art. Such tumor cell lines include, for example, HTB-104 or CRL-1973 cells (testicular tumor cells; available from the American Type Culture Collection); or BT483, Hs578T, HTB2, BT20 or T47D cells (breast cancer cell lines). Other cancer cell lines known to those in the art may be used, as well.

The nature of the matrix on which the vessel rings and/or stem or tumor cells are cultured is important for successful angiogenesis. Therefore, a preferred embodiment of the invention is for these tissues and cells to be cultured on plates or dishes that have been prepared with a physiological gel to create a growth matrix. Preferably, this growth matrix comprises non-denatured human collagen. In another preferred embodiment, the physiological gel is fibrin, collagen or MATRIGEL®. More preferably the gel is fibrin.

Any substance, or combination of substances that is suspected of angiogenesis modulation activity may be screened by the method. This includes purified preparations of compounds and various extracts such as plant or animal tissue extracts or may be from a microorganism. Accordingly, such substances may have to be brought into a suitable form for administration to the nonembryonic pluripotent stem cells. Those skilled in the art will be familiar with various methods for bringing such substances into suitable form for administration.

In another preferred embodiment, when the method is used to test compounds for angiogenesis enhancement, the medium is substantially serum free such that whole serum is absent and the medium has no serum constituents or a minimal number of constituents from serum or other sources that are necessary for angiogenesis.

In another preferred embodiment, after the substance is administered, the nonembryonic pluripotent stem cells are cultured for a time sufficient to allow clear prevention and/or regression of new blood vessel growth, such as, for example, 7 to 14 days after the substance is administered. The state of the new blood vessel growth is then compared to the recorded response and preferably a control.

5.1.2 Characterization of Angiogenesis

In accordance with the present invention, angiogenesis may be measured by identification of cell surface markers, using standard techniques in the art, such as immunocytochemistry. In accordance with this aspect of the invention, samples demonstrating detectable angiogenic responses (i.e., new vascular growth) may be assayed using immunohistochemistry. Examples of antibodies that may be used include monoclonal mouse anti-human factor VIII related antigen (Dako, Denmark), an anti-human endothelial cell mAb (Gibco, Grand Island, N.Y.) and a CD31-specific mAb (clone 20G5) produced in the John Curtin School of Medical Research. Immunohistochemical staining of angiogenic samples may be performed to detect Factor VIII related antigen, a reaction that clearly demonstrates that the outgrowths are blood vessels. The vessels also reacted with a mAb specific for human endothelial cells (Gibco) and with a mAb to CD31, an antigen only expressed on endothelial cells, platelets and some leukocytes. Examination of angiogenic samples under the electron microscope can also be performed to reveal cells with a classic endothelial morphology.

Following culture for 7 to 21 days, angiogenesis is quantified and compared with control cultures. In the case of putative anti-angiogenic substances, a reduced growth of blood vessels compared with the control cultures will be determined. The invention also encompasses assaying test substances for their ability to induce regression of recently formed blood vessels by adding the test substance to established angiogenesis responses (i.e., after 7-21 days of culture) and monitoring “die-back” of blood vessels microscopically for the next 7-14 days.

In certain embodiments, angiogenesis may be identified by characterizing differentially expressed genes (for example, characterizing a pool of genes from an undifferentiated progenitor cell(s) of interest versus a pool of genes from a differentiated cell derived from the progenitor cell). For example, nucleic acid amplification methods such as polymerase chain reaction (PCR) or transcription-based amplification methods (e.g., in vitro transcription (IVT)) may be used to profile gene expression in different populations of cells, e.g., by use of a polynucleotide microarray. Such methods to profile differential gene expression are well known in the art (see, e.g., Wieland et al., 1990, Proc. Natl. Acad. Sci. USA 87: 2720-2724; Lisitsyn et al., 1993, Science 259: 946-951; Lisitsyn et al., 1995, Meth. Enzymology 254: 291-304; U.S. Pat. No. 5,436,142; U.S. Pat. No. 5,501,964; Lisitsyn et al., 1994, Nature Genetics 6: 57-63; Hubank and Schatz, 1994, Nucleic Acids Research 22: 5640-5648; Zeng et al., 1994, Nucleic Acids Research 22: 4381-4385; U.S. Pat. No. 5,525,471; Linsley et al., U.S. Pat. No. 6,271,002, entitled “RNA amplification method,” issued Aug. 7, 2001; Van Gelder et al., U.S. Pat. No. 5,716,785, entitled “Processes for genetic manipulations using promoters,” issued Feb. 10, 1998; Stoflet et al., 1988, Science 239: 491-494, 1988; Sarkar and Sommer, 1989, Science 244: 331-334; Mullis et al., U.S. Pat. No. 4,683,195; Malek et al., U.S. Pat. No. 5,130,238; Kacian and Fultz, U.S. Pat. No. 5,399,491; Burg et al., U.S. Pat. No. 5,437,990; Van Gelder et al., 1990, Proc. Natl. Acad. Sci. USA 87: 1663; Lockhart et al., 1996, Nature Biotechnol. 14, 1675; Shannon, U.S. Pat. No. 6,132,997; Lindemann et al., U.S. Pat. No. 6,235,503, entitled “Procedure for subtractive hybridization and difference analysis,” issued May 22, 2001).

Commercially available kits are available for gene profiling, e.g., the display PROFILE™ series of kits (Qbiogene, Carlsbad, Calif., which uses a gel-based approach for profiling gene expression. The kits utilize Restriction Fragment Differential Display-PCR (RFDD-PCR) to compare gene expression patterns in eukaryotic cells. A PCR-Select Subtraction Kit (Clontech) and a PCR-Select Differential Screening Kit (Clontech) may also be used, which permits identification of differentially expressed clones in a subtracted library. After generating pools of differentially expressed genes with the PCR-Select Subtraction kit, the PCR-Select Differential Screening kit is used. The subtracted library is hybridized with probes synthesized directly from tester and driver populations, a probe made from the subtracted cDNA, and a probe made from reverse-subtracted cDNA (a second subtraction performed in reverse). Clones that hybridize to tester but not driver probes are differentially expressed; however, non-subtracted probes are not sensitive enough to detect rare messages. Subtracted probes are greatly enriched for differentially expressed cDNAs, but may give false positive results. Using both subtracted and non-subtracted probes according to the manufacturer's (Clontech) instructions identifies differentially expressed genes.

5.2 The Compounds of the Invention

Examples of test compounds which may be screened for modulation of angiogenesis include, but are not limited to, small molecules, organic compounds, inorganic compounds, polypeptides, peptides, proteins, hormones, cytokines, oligonucleotides, nucleic acids or other macromolecules.

The term “compound” as used herein describes any molecule, e.g., a protein or non-protein organic pharmaceutical. Generally, a plurality of assay mixtures is run in parallel with different compound concentrations to obtain a differential response to the various concentrations. Typically, one of these concentrations serves as a negative control, i.e., at zero concentration or below the level of detection.

Candidate compounds encompass numerous chemical classes, though typically they are organic molecules, preferably small organic compounds having a molecular weight of more than 50 and less than about 2,500 daltons. Candidate compounds comprise functional groups necessary for structural interaction with proteins, particularly hydrogen bonding, and typically include at least an amine, carbonyl, hydroxyl or carboxyl group, preferably at least two of the functional chemical groups. The candidate compounds often comprise cyclical carbon on heterocyclic structures and/or aromatic or polyaromatic structures substituted with one or more of the above functional groups. Candidate compounds are also found among biomolecules including, but not limited to: peptides, saccharides, fatty acids, steroids, purines, pyrimidines, derivatives, structural analogs or combinations thereof.

Candidate modulatory compounds are obtained from a wide variety of sources including libraries of synthetic or natural compounds. For example, numerous means are available for random and directed synthesis of a wide variety of organic compounds and biomolecules, including expression of randomized oligonucleotides and oligopeptides. Alternatively, libraries of natural compounds in the form of bacterial, fungal, plant and animal extracts are available or readily produced. Additionally, natural or synthetically produced libraries and compounds are readily modified through conventional chemical, physical and biochemical means, and may be used to produce combinatorial libraries. Known pharmacological agents may be subjected to directed or random chemical modifications, such as acylation, alkylation, esterification, amidification, etc. to produce structural analogs. New potential therapeutic agents may also be created using methods such as rational drug design or computer modelling. Screening may be directed to known pharmacologically active compounds and chemical analogs thereof, or to new compounds with unknown properties such as those created through rational drug design.

5.2.1 TNF-α Inhibitors

Members of one class of compounds have been identified, using the assay methods disclosed elsewhere herein, as modulating angiogenesis and/or vasogenesis; specifically, these compounds are anti-angiogenic compounds; more specifically, these compounds include IMiDs™ (Celgene Corporation). As used herein and unless otherwise indicated, the term “anti-angiogenic compounds” or “IMiDs™” used herein encompasses small organic molecules that markedly inhibit TNF-α, and have anti-angiogenic activity; that is, they act to inhibit the formation of new blood vessels. Specifically, the anti-angiogenic compounds of the invention enhance the degradation of TNF-α mRNA. This class includes racemic, stereomerically enriched or stereomerically pure and pharmaceutically acceptable salts, solvates, hydrates, stereoisomers, clathrates, and prodrugs of these anti-angiogenic compounds. Preferred compounds used in the invention are small organic molecules having a molecular weight less than about 1000 g/mol, and are not proteins, peptides, oligonucleotides, oligosaccharides or other macromolecules. Specific compounds of the invention are discussed below. These compounds can be obtained commercially from Celgene (Warren, N.J.), or may be prepared in accordance with the methods described in the patents or publications listed herein.

Specific examples of anti-angiogenic compounds of the invention, include, but are not limited to, cyano and carboxy derivatives of substituted styrenes such as those disclosed in U.S. Pat. No. 5,929,117; 1-oxo-2-(2,6-dioxo-3-fluoropiperidin-3yl)isoindolines and 1,3-dioxo-2-(2,6-dioxo-3-fluoropiperidine-3-yl)isoindolines such as those described in U.S. Pat. No. 5,874,448; the tetra substituted 2-(2,6-dioxopiperdin-3-yl)-1-oxoisoindolines described in U.S. Pat. No. 5,798,368; 1-oxo and 1,3-dioxo-2-(2,6-dioxopiperidin-3-yl) isoindolines (e.g., 4-methyl derivatives of thalidomide and EM-12), including, but not limited to, those disclosed in U.S. Pat. No. 5,635,517; and a class of non-polypeptide cyclic amides disclosed in U.S. Pat. Nos. 5,698,579 and 5,877,200. The entirety of each of the patents identified herein are incorporated herein by reference. Anti-angiogenic compounds of the invention do not, however, include thalidomide.

Other specific anti-angiogenic compounds of the invention include, but are not limited to, 1-oxo-and 1,3 dioxo-2-(2,6-dioxopiperidin-3-yl)isoindolines substituted with amino or substituted amino in the benzo ring as described in U.S. Pat. No. 5,635,517 which is incorporated herein. These compounds have the structure I: 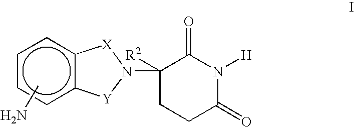
in which one of X and Y is C═O, the other of X and Y is C═O or CH₂, and R² is hydrogen or lower alkyl, in particular methyl. Specific anti-angiogenic compounds include, but are not limited to:
1-oxo-2-(2,6-dioxopiperidin-3-yl)-4-aminoisoindoline;
1-oxo-2-(2,6-dioxopiperidin-3-yl)-5-aminoisoindoline;
1-oxo-2-(2,6-dioxopiperidin-3-yl)-6-aminoisoindoline;
1-oxo-2-(2,6-dioxopiperidin-3-yl)-7-aminoisoindoline;
1,3-dioxo-2-(2,6-dioxopiperidin-3-yl)-4-aminoisoindoline;
and 1,3-dioxo-2-(2,6-dioxopiperidin-3-yl)-5-aminoisoindoline.

Other specific anti-angiogenic compounds of the invention belong to a class of substituted 2-(2,6-dioxopiperidin-3-yl) phthalimides and substituted 2-(2,6-dioxopiperidin-3-yl)-1-oxoisoindoles, such as those described in U.S. Pat. Nos. 6,281,230; 6,316,471; 6,335,349; and 6,476,052, and International Patent Application No. PCT/US97/13375 (International Publication No. WO 98/03502), each of which is incorporated herein by reference in its entirety. Compounds representative of this class are of the formulas: 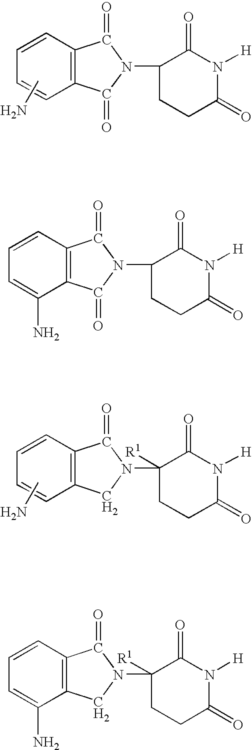
wherein R¹ is hydrogen or methyl. In a separate embodiment, the invention encompasses the use of enantiomerically pure forms (e.g. optically pure (R) or (S) enantiomers) of these compounds.

Still other specific anti-angiogenic compounds of the invention belong to a class of isoindole-imides disclosed in U.S. patent application Ser. Nos. 10/032,286 and 09/972,487, and International Application No. PCT/US01/50401 (International Publication No. WO 02/059106), each of which is incorporated herein by reference in its entirety. Representative compounds are of formula II: 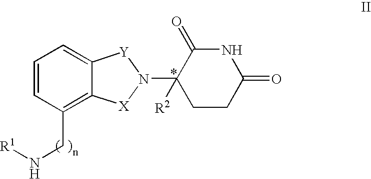
and pharmaceutically acceptable salts, hydrates, solvates, clathrates, enantiomers, diastereomers, racemates, and mixtures of stereoisomers thereof, wherein:
one of X and Y is C═O and the other is CH₂ or C═O;
R¹ is H, (C₁-C₈)alkyl, (C₃-C₇)cycloalkyl, (C₂-C₈)alkenyl, (C₂-C₈)alkynyl, benzyl, aryl, (C₀-C₄)alkyl-(C₁-C₆)heterocycloalkyl, (C₀-C₄)alkyl C₂-C₅)heteroaryl, C(O)R³, C(S)R³, C(O)OR⁴, (C₁-C₈)alkyl-N(R⁶)₂, (C₁-C₅)alkyl-OR⁵, (C₁-C₈)alkyl-C(O)OR⁵, C(O)NHR³, C(S)NHR³, C(O)NR³R^{3′}, C(S)NR³R^{3′} or (C₁-C₈)alkyl-O(CO)R⁵;
R² is H, F, benzyl, (C₁-C₈)alkyl, (C₂-C₈)alkenyl, or (C₂-C₈)alkynyl;
R³ and R^{3′} are independently (C₁-C₈)alkyl, (C₃-C₇)cycloalkyl, (C₂-C₈)alkenyl, (C₂-C₈)alkynyl, benzyl, aryl, (C₀-C₄)alkyl-(C₁-C₆)heterocycloalkyl, (C₀-C₄)alkyl-(C₂-C₅)heteroaryl, (C₀-C₈)alkyl-N(R⁶)₂, (C₁-C₈)alkyl-OR⁵, (C₁-C₈)alkyl-C(O)OR⁵, (C₁-C₈)alkyl-O(CO)R⁵, or C(O)OR⁵;
R⁴ is (C₁-C₈)alkyl, (C₂-C₈)alkenyl, (C₂-C₈)alkynyl, (C₁-C₄)alkyl-OR⁵, benzyl, aryl, (C₀-C₄)alkyl-(C₁-C₆)heterocycloalkyl, or (C₀-C₄)alkyl-(C₂-C₅)heteroaryl;
R⁵ is (C₁-C₈)alkyl, (C₂-C₈)alkenyl, (C₂-C₈)alkynyl, benzyl, aryl, or (C₂-C₅)heteroaryl;
each occurrence of R⁶ is independently H, (C₁-C₈)alkyl, (C₂-C₈)alkenyl, (C₂-C₈)alkynyl, benzyl, aryl, (C₂-C₅)heteroaryl, or (C₀-C₈)alkyl-C(O)O—R⁵ or the R⁶ groups can join to form a heterocycloalkyl group;
n is 0 or 1; and
* represents a chiral-carbon center.

In specific compounds of formula II, when n is 0 then R¹ is (C₃-C₇)cycloalkyl, (C₂-C₈)alkenyl, (C₂-C₈)alkynyl, benzyl, aryl, (C₀-C₄)alkyl-(C₁-C₆)heterocycloalkyl, (C₀-C₄)alkyl-(C₂-C₅)heteroaryl, C(O)R³, C(O)OR⁴, (C₁-C₈)alkyl-N(R⁶)₂, (C₁-C₈)alkyl-OR⁵, (C₁-C₈)alkyl-C(O)OR⁵, C(S)NHR³, or (C₁-C₈)alkyl-O(CO)R⁵;
R² is H or (C₁-C₈)alkyl; and
R³ is (C₁-C₈)alkyl, (C₃-C₇)cycloalkyl, (C₂-C₈)alkenyl, (C₂-C₈)alkynyl, benzyl, aryl, (C₀-C₄)alkyl-(C₁-C₆)heterocycloalkyl, (C₀-C₄)alkyl-(C₂-C₅)heteroaryl, (C₅-C₈)alkyl-N(R⁶)₂; (C₀-C₈)alkyl-NH—C(O)O—R⁵; (C₁-C₈)alkyl-OR⁵, (C₁-C₈)alkyl-C(O)OR⁵, (C₁-C₈)alkyl-O(CO)R⁵, or C(O)OR⁵; and the other variables have the same definitions.

In other specific compounds of formula II, R² is H or (C₁-C₄)alkyl.

In other specific compounds of formula II, R¹ is (C₁-C₈)alkyl or benzyl.

In other specific compounds of formula II, R¹ is H, (C₁-C₈)alkyl, benzyl, CH₂OCH₃, CH₂CH₂OCH₃, or

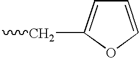

In another embodiment of the compounds of formula II, R¹ is 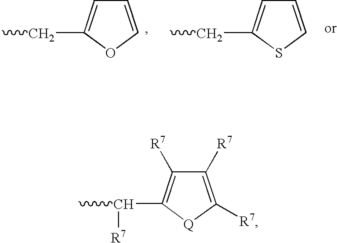
wherein Q is O or S, and each occurrence of R⁷ is independently H, (C₁-C₈)alkyl, benzyl, CH₂OCH₃, or CH₂CH₂OCH₃.

In other specific compounds of formula II, R¹ is C(O)R³.

In other specific compounds of formula II, R³ is (C₀-C₄)alkyl-(C₂-C₅)heteroaryl, (C₁-C₅)alkyl, aryl, or (C₀-C₄)alkyl-OR⁵.

In other specific compounds of formula II, heteroaryl is pyridyl, furyl, or thienyl.

In other specific compounds of formula II, R¹ is C(O)OR⁴.

In other specific compounds of formula II, the H of C(O)NHC(O) can be replaced with (C₁-C₄)alkyl, aryl, or benzyl.

Still other specific anti-angiogenic compounds of the invention belong to a class of isoindole-imides disclosed in U.S. patent application Ser. No. 09/781,179, International Publication No. WO 98/54170, and U.S. Pat. No. 6,395,754, each of which are incorporated herein by reference. Representative compounds are of formula III: 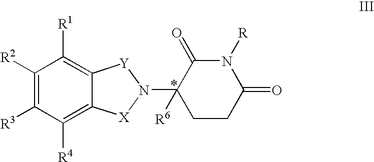
and pharmaceutically acceptable salts, hydrates, solvates, clathrates, enantiomers, diastereomers, racemates, and mixtures of stereoisomers thereof, wherein:
one of X and Y is C═O and the other is CH₂ or C═O;
R is H or CH2OCOR′;
(i) each of R¹, R², R³, or R⁴, independently of the others, is halo, alkyl of 1 to 4 carbon atoms, or alkoxy of 1 to 4 carbon atoms or (ii) one of R¹, R², R³, or R⁴ is nitro or —NHR⁵ and the remaining of R¹, R², R³, or R⁴ are hydrogen;
R⁵ is hydrogen or alkyl of 1 to 8 carbons
R⁶ hydrogen, alkyl of 1 to 8 carbon atoms, benzo, chloro, or fluoro;
R′ is R⁷—CHR¹⁰—N(R⁸R⁹);
R⁷ is m-phenylene or p-phenylene or —(CₙH₂ₙ)— in which n has a value of 0 to 4;
each of R8 and R9 taken independently of the other is hydrogen or alkyl of 1 to 8 carbon atoms, or R8 and R9 taken together are tetramethylene, pentamethylene, hexamethylene, or —CH₂CH₂[X]X₁CH₂CH₂— in which [X]X₁ is —O—, —S—, or —NH—;
R¹⁰ is hydrogen, alkyl of to 8 carbon atoms, or phenyl; and
* represents a chiral-carbon center.

The most preferred anti-angiogenic compounds of the invention are 4-(amino)-2-(2,6-dioxo(3-piperidyl))-isoindoline-1,3-dione and 3-(4-amino-1-oxo-1,3-dihydro-isoindol-2-yl)-piperidine-2,6-dione. The compounds can be obtained via standard, synthetic methods (see e.g., U.S. Pat. No. 5,635,517, incorporated herein by reference). Certain of these compounds, such as thalidomide may be commercially available (e.g. Thalomid™, Actimid™, and Revimid™ (Celgene, Inc., Warren, N.J.)). 4-(Amino)-2-(2,6-dioxo(3-piperidyl))-isoindoline-1,3-dione (ACTIMID™) has the following chemical structure: 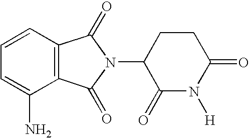
3-(4-amino-1-oxo-1,3-dihydro-isoindol-2-yl)-piperidine-2,6-dione (REVIMID™) has the following chemical structure:

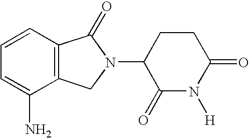

Other above compounds can be made by methods known in the art, including those disclosed in the patents cited above which are incorporated by reference in their entries.

Clearly, the most preferred compound of the invention is thalidomide, aminothalidomide, and 3-(4-Amino-1-oxo-1,3-dihydro-isoindol-2-yl)-piperidine-2,6-dione.

The compounds of the invention can be assayed for their ability to modulate the production of TNF-α using methods well known in the art, for example, those assays disclosed in Robarge et al., U.S. application publication serial no. U.S. 2003045552, published Mar. 6, 2003, entitled “Isoindole-Imide Compounds, Compositions, And Uses Thereof,” which is incorporated herein by reference in its entirety.

As used herein and unless otherwise indicated, the term “stereomerically pure” means a composition that comprises one stereoisomer of a compound and is substantially free of other stereoisomers of that compound. For example, a stereomerically pure composition of a compound having one chiral center will be substantially free of the opposite enantiomer of the compound. A stereomerically pure composition of a compound having two chiral centers will be substantially free of other diastereomers of the compound. As used herein and unless otherwise indicated, the term “enantiomerically pure” means a stereomerically pure composition of a compound having one chiral center. As used herein and unless otherwise indicated, the term “stereomerically enriched” means a composition that comprises greater than about 60% by weight of one stereoisomer of a compound, preferably greater than about 70% by weight, more preferably greater than about 80% by weight of one stereoisomer of a compound. As used herein, the term “enantiomerically pure” means a stereomerically pure composition of a compound having one chiral center. Similarly, the term “enantiomerically enriched” means a stereomerically enriched composition of a compound having one chiral center.

5.2.2 PDE IV Inhibitors

Another class of compounds expected to have anti-angiogenic activity is referred to as PDE IV inhibitors. PDE IV inhibitors, like IMiDs, have TNF-α inhibitory activity. Preferred compounds used in the invention are known Selective Cytokine Inhibitory Drugs (SelCIDs™) of Celgene Corporation. Members of this class of compounds may also be tested for angiogenesis modulatory activity.

As used herein and unless otherwise indicated, the term “SeICIDS™” used in the invention encompasses small molecule drugs, e.g., small organic molecules which are not peptides, proteins, nucleic acids, oligosaccharides or other macromolecules. Preferred compounds inhibit TNF-α production. Further, the compounds may also have a modest inhibitory effect on LPS induced IL1β and IL12.

More preferably, the compounds of the invention are potent PDE IV inhibitors. PDE IV is one of the major phosphodiesterase isoenzymes found in human myeloid and lymphoid lineage cells. The enzyme plays a crucial part in regulating cellular activity by degrading the ubiquitous second messenger cAMP and maintaining it at low intracellular levels.

Specific examples of selective cytokine inhibitory drugs include, but are not limited to, the cyclic imides disclosed in U.S. Pat. No. 5,605,914; the cycloalkyl amides and cycloalkyl nitriles of U.S. Pat. Nos. 5,728,844 and 5,728,845, respectively; the aryl amides (for example, an embodiment being N-benzoyl-3-amino-3-(3′,4′-dimethoxyphenyl)-propanamide) of U.S. Pat. Nos. 5,801,195 and 5,736,570; the imide/amide ethers and alcohols (for example 3-phthalimido-3-(3′,4′-dimethoxypheryl)propan-1-ol) disclosed in U.S. Pat. No. 5,703,098; the succinimides and maleimides (for example methyl 3-(3′,4′,5′6′-petrahydrophthalimdo)-3-(3″,4″-dimethoxyphenyl)propionate) disclosed in U.S. Pat. No. 5,658,940; imido and amido substituted alkanohydroxamic acids disclosed in WO 99/06041 and substituted phenethylsulfones disclosed in U.S. Pat. No. 6,020,358; and aryl amides such as N-benzoyl-3-amino-3-(3′,4′-dimethoxyphenyl)propanamide as described in U.S. Pat. No. 6,046,221. The entireties of each of the patents and patent applications identified herein are incorporated herein by reference.

Additional selective cytokine inhibitory drugs belong to a family of synthesized chemical compounds of which typical embodiments include 3-(1,3-dioxobenzo-[f]isoindol-2-yl)-3-(3-cyclopentyloxy-4-methoxyphenyl)propionamide and 3-(1,3-dioxo-4-azaisoindol-2-yl)-3-(3,4-dimethoxyphenyl)-propionamide.

Other specific selective cytokine inhibitory drugs belong to a class of non-polypeptide cyclic amides disclosed in U.S. Pat. Nos. 5,698,579 and 5,877,200, both of which are incorporated herein. Representative cyclic amides include compounds of the formula: 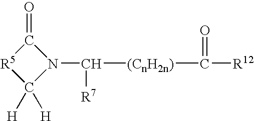
wherein n has a value of 1, 2, or 3;
R⁵ is o-phenylene, unsubstituted or substituted with 1 to 4 substituents each selected independently from the group consisting of nitro, cyano, trifluoromethyl, carbethoxy, carbomethoxy, carbopropoxy, acetyl, carbamoyl, acetoxy, carboxy, hydroxy, amino, alkylamino, dialkylamino, acylamino, alkyl of 1 to 10 carbon atoms, alkyl of 1 to 10 carbon atoms, and halo;
R⁷ is (i) phenyl or phenyl substituted with one or more substituents each selected independently of the other from the group consisting of nitro, cyano, trifluoromethyl, carbethoxy, carbomethoxy, carbopropoxy, acetyl, carbamoyl, acetoxy, carboxy, hydroxy, amino, alkyl of 1 to 10 carbon atoms, alkoxy of 1 to 10 carbon atoms, and halo, (ii) benzyl unsubstituted or substituted with 1 to 3 substituents selected from the group consisting of nitro, cyano, trifluoromethyl, carbothoxy, carbomethoxy, carbopropoxy, acetyl, carbamoyl, acetoxy, carboxy, hydroxy, amino, alkyl of 1 to 10 carbon atoms, alkoxy of 1 to 10 carbon atoms, and halo, (iii) naphthyl, and (iv) benzyloxy;
R¹² is —OH, alkoxy of 1 to 12 carbon atoms, or 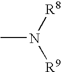
R⁸ is hydrogen or alkyl of 1 to 10 carbon atoms; and
R⁹ is hydrogen, alkyl of 1 to 10 carbon atoms, —COR¹⁰, or —SO₂R¹⁰, wherein R¹⁰ is hydrogen, alkyl of 1 to 10 carbon atoms, or phenyl.

Specific compounds of this class include, but are not limited to:
3-phenyl-2-(1-oxoisoindolin-2-yl)propionic acid;
3-phenyl-2-(1-oxoisoindolin-2-yl)propionamide;
3-phenyl-3-(1-oxoisoindolin-2-yl)propionic acid;
3-phenyl-3-(1-oxoisoindolin-2-yl)propionamide;
3-(4-methoxyphenyl)-3-(1-oxisoindolin-yl)propionic acid;
3-(4-methoxyphenyl)-3-(1-oxisoindolin-yl)propionamide;
3-(3,4-dimethoxyphenyl)-3-(1-oxisoindolin-2-yl)propionic acid;
3-(3,4-dimethoxy-phenyl)-3-(1-oxo-1,3-dihydroisoindol-2-yl)-propionamide;
3-(3,4-dimethoxyphenyl)-3-(1-oxisoindolin-2-yl)propionamide;
3-(3,4-diethoxyphenyl)-3-(1-oxoisoindolin-yl)propionic acid;
methyl 3-(1-oxoisoindolin-2-yl)-3-(3-ethoxy-4-methoxyphenyl)propionate;
3-(1-oxoisoindolin-2-yl)-3-(3-ethoxy-4-methoxyphenyl)propionic acid;
3-(1-oxoisoindolin-2-yl)-3-(3-propoxy-4-methoxyphenyl)propionic acid;
3-(1-oxoisoindolin-2-yl)-3-(3-butoxy-4-methoxyphenyl)propionic acid;
3-(1-oxoisoindolin-2-yl)-3-(3-propoxy-4-methoxyphenyl)propionamide;
3-(1-oxoisoindolin-2-yl)-3-(3-butoxy-4-methoxyphenyl)propionamide;
methyl 3-(1-oxoisoindolin-2-yl)-3-(3-butoxy-4-methoxyphenyl)propionate; and
methyl 3-(1-oxoisoindolin-2-yl)-3-(3-propoxy-4-methoxyphenyl)propionate.

Other specific selective cytokine inhibitory drugs include the imido and amido substituted alkanohydroxamic acids disclosed in WO 99/06041, which is incorporated herein by reference. Examples of such compound include, but are not limited to: 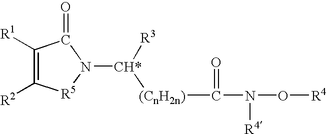
wherein each of R¹ and R², when taken independently of each other, is hydrogen, lower alkyl, or R¹ and R², when taken together with the depicted carbon atoms to which each is bound, is o-phenylene, o-naphthylene, or cyclohexene-1,2-diyl, unsubstituted or substituted with 1 to 4 substituents each selected independently from the group consisting of nitro, cyano, trifluoromethyl, carbethoxy, carbomethoxy, carbopropoxy, acetyl, carbamoyl, acetoxy, carboxy, hydroxy, amino, alkylamino, dialkylamino, acylamino, alkyl of 1 to 10 carbon atoms, alkoxy of 1 to 10 carbon atoms, and halo;
R³ is phenyl substituted with from one to four substituents selected from the group consisting of nitro, cyano, trifluoromethyl, carbethoxy, carbomethoxy, carbopropoxy, acetyl, carbamoyl, acetoxy, carboxy, hydroxy, amino, alkyl of 1 to 10 carbon atoms, alkoxy of 1 to 10 carbon atoms, alkylthio of 1 to 10 carbon atoms, benzyloxy, cycloalkoxy of 3 to 6 carbon atoms, C₄-C₆-cycloalkylidenemethyl, C₃-C₁₀-alkylidenemethyl, indanyloxy, and halo;
R⁴ is hydrogen, alkyl of 1 to 6 carbon atoms, phenyl, or benzyl;
R^{4′} is hydrogen or alkyl of 1 to 6 carbon atoms;
R⁵ is —CH₂—, —CH₂—CO—, —SO₂—, —S—, or —NHCO—;
n has a value of 0, 1, or 2; and
the acid addition salts of said compounds which contain a nitrogen atom capable of being protonated.

Additional specific selective cytokine inhibitory drugs used in the invention include, but are not limited to:
3-(3-ethoxy-4-methoxyphenyl)-N-hydroxy-3-(1-oxoisoindolinyl)propionamide;
3-(3-ethoxy-4-methoxyphenyl)-N-methoxy-3-(1-oxoisoindolinyl)propionamide;
N-benzyloxy-3-(3-ethoxy-4-methoxyphenyl)-3-phthalimidopropionamide;
N-benzyloxy-3-(3-ethoxy-4-methoxyphenyl)-3-(3-nitrophthalimido)propionamide;
N-benzyloxy-3-(3-ethoxy-4-methoxyphenyl)-3-(1-oxoisoindolinyl)propionamide;
3-(3-ethoxy-4-methoxyphenyl)-N-hydroxy-3-phthalimidopropionamide;
N-hydroxy-3-(3,4-dimethoxyphenyl)-3-phthalimidopropionamide;
3-(3-ethoxy-4-methoxyphenyl)-N-hydroxy-3-(3-nitrophthalimido)propionamide;
N-hydroxy-3-(3,4-dimethoxyphenyl)-3-(1-oxoisoindolinyl)propionamide;
3-(3-ethoxy-4-methoxyphenyl)-N-hydroxy-3-(4-methyl-phthalimido)propionamide;
3-(3-cyclopentyloxy-4-methoxyphenyl)-N-hydroxy-3-phthalimidopropionamide;
3-(3-ethoxy-4-methoxyphenyl)-N-hydroxy-3-(1,3-dioxo-2,3-dihydro-1H-benzo[f]isoindol-2-yl)propionamide;
N-hydroxy-3-{3-(2-propoxy)-4-methoxyphenyl}-3-phthalimidopropionamide;
3-(3-ethoxy-4-methoxyphenyl)-3-(3,6-difluorophthalimido)-N-hydroxypropionamide;
3-(4-aminophthalimido)-3-(3-ethoxy-4-methoxyphenyl)-N-hydroxypropionamide;
3-(3-aminophthalimido)-3-(3-ethoxy-4-methoxyphenyl)-N-hydroxypropionamide;
N-hydroxy-3-(3,4-dimethoxyphenyl)-3-(1-oxoisoindolinyl)propionamide;
3-(3-cyclopentyloxy-4-methoxyphenyl)-N-hydroxy-3-(1-oxoisoindolinyl)propionamide; and
N-benzyloxy-3-(3-ethoxy-4-methoxyphenyl)-3-(3-nitrophthalimido)propionamide.

Additional selective cytokine inhibitory drugs used in the invention include the substituted phenethylsulfones substituted on the phenyl group with a oxoisoindine group. Examples of such compounds include, but are not limited to, those disclosed in U.S. Pat. No. 6,020,358, which is incorporated herein, which include the following: 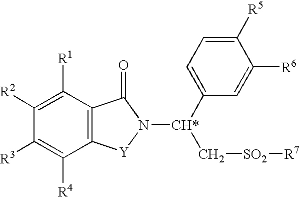
wherein the carbon atom designated * constitutes a center of chirality;
Y is C═O, CH2, SO₂, or CH₂C═O; each of R¹, R², R³, and R⁴, independently of the others, is hydrogen, halo, alkyl of 1 to 4 carbon atoms, alkoxy of 1 to 4 carbon atoms, nitro, cyano, hydroxy, or —NR⁸R⁹; or any two of R¹, R², R³, and R⁴ on adjacent carbon atoms, together with the depicted phenylene ring are naphthylidene;
each of R⁵ and R⁶, independently of the other, is hydrogen, alkyl of 1 to 4 carbon atoms, alkoxy of 1 to 4 carbon atoms, cyano, or cycloalkoxy of up to 18 carbon atoms;
R⁷ is hydroxy, alkyl of 1 to 8 carbon atoms, phenyl, benzyl, or NR^{8′} R^{9′};
each of R⁸ and R⁹ taken independently of the other is hydrogen, alkyl of 1 to 8 carbon atoms, phenyl, or benzyl, or one of R⁸ and R⁹ is hydrogen and the other is —COR¹⁰ or —SO₂R¹⁰, or R⁸ and R⁹ taken together are tetramethylene, pentamethylene, hexamethylene, or —CH₂CH₂X¹CH₂CH₂— in which X¹ is —O—, —S— or —NH—; and
each of R^{8′} and R^{9′} taken independently of the other is hydrogen, alkyl of 1 to 8 carbon atoms, phenyl, or benzyl, or one of R^{8′} and R^{9′} is hydrogen and the other is —COR^{10′} or —SO₂R^{10′}, or R^{8′} and R^{9′} taken together are tetramethylene, pentamethylene, hexamethylene, or —CH₂CH₂X²CH₂CH₂— in which X² is —O—, —S—, or —NH—.

It will be appreciated that while for convenience the above compounds are identified as phenethylsulfones, they include sulfonamides when R⁷ is NR^{8′}R^{9′}.

Specific groups of such compounds are those in which Y is C═O or CH₂.

A further specific group of such compounds are those in which each of R¹, R², R³, and R⁴ independently of the others, is hydrogen, halo, methyl, ethyl, methoxy, ethoxy, nitro, cyano, hydroxy, or —NR⁸R⁹ in which each of R⁸ and R⁹ taken independently of the other is hydrogen or methyl or one of R⁸ and R⁹ is hydrogen and the other is —COCH₃.

Particular compounds are those in which one of R¹, R², R³, and R⁴ is —NH₂ and the remaining of R¹, R², R³, and R⁴ are hydrogen.

Particular compounds are those in which one of R¹, R², R³, and R⁴ is —NHCOCH₃ and the remaining of R¹, R², R³, and R⁴ are hydrogen.

Particular compounds are those in which one of R¹, R², R³, and R⁴ is —N(CH₃)₂ and the remaining of R¹, R², R³, and R⁴ are hydrogen.

A further preferred group of such compounds are those in which one of R¹, R², R³, and R⁴ is methyl and the remaining of R¹, R², R³, and R⁴ are hydrogen.

Particular compounds are those in which one of R¹, R², R³, and R⁴ is fluoro and the remaining of R¹, R², R³, and R⁴ are hydrogen.

Particular compounds are those in which each of R⁵ and R⁶, independently of the other, is hydrogen, methyl, ethyl, propyl, methoxy, ethoxy, propoxy, cyclopentoxy, or cyclohexoxy.

Particular compounds are those in which R⁵ is methoxy and R⁶ is monocycloalkoxy, polycycloalkoxy, and benzocycloalkoxy.

Particular compounds are those in which R⁵ is methoxy and R⁶ is ethoxy.

Particular compounds are those in which R⁷ is hydroxy, methyl, ethyl, phenyl, benzyl, or NR^{8′}R^{9′} in which each of R^{8′} and R^{9′} taken independently of the other is hydrogen or methyl.

Particular compounds are those in which R⁷ is methyl, ethyl, phenyl, benzyl or NR^{8′} R^{9′} in which each of R^{8′} and R^{9′} taken independently of the other is hydrogen or methyl.

Particular compounds are those in which R⁷ is methyl.

Particular compounds are those in which R⁷ is NR^{8′} R^{9′} in which each of R^{8′} and R^{9′} taken independently of the other is hydrogen or methyl.

Other specific selective cytokine inhibitory drugs include fluoroalkoxy-substituted 1,3-dihydro-isoindolyl compounds found in U.S. Provisional Application No. 60/436,975 to G. Muller et al., filed Dec. 30, 2002, which is incorporated herein in its entirety by reference. Representative fluoroalkoxy-substituted 1,3-dihydro-isoindolyl compounds include compounds of the formula: 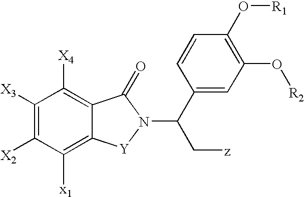
wherein:
Y is —C(O)—, —CH₂, —CH₂C(O)—, —C(O)CH₂—, or SO₂;
Z is —H, —C(O)R³, —(C₀₋₁-alkyl)-SO₂—(C₁₋₄-alkyl), —C₁₋₈-alkyl, —CH₂OH, CH₂(O)(C₁₋₈-alkyl) or —CN;
R₁ and R₂ are each independently —CHF₂, —C₁₋₈-alkyl, —C₃₋₁₈-cycloalkyl, or —(C₁₋₁₀-alkyl)(C₃₋₁₈-cycloalkyl), and at least one of R₁ and R₂ is CHF₂;
R³ is —NR⁴R⁵, -alkyl, —OH, —O-alkyl, phenyl, benzyl, substituted phenyl, or substituted benzyl;
R⁴ and R⁵ are each independently —H, —C₁₋₈-alkyl, —OH, —OC(O)R⁶;
R⁶ is —C₁₋₈-alkyl, -amino(C₁₋₈-alkyl), -phenyl, -benzyl, or -aryl;
X₁, X₂, X₃, and X₄ are each independent —H, -halogen, -nitro, —NH₂, —CF₃, —C₁₋₆-alkyl, —(C₀₋₄-alkyl)-(C₃₋₆-cycloalkyl), (C₀₋₄-alkyl)-NR⁷R⁸, (C₀₋₄-alkyl)-N(H)C(O)—(R⁸), (C₀₋₄-alkyl)-N(H)C(O)N(R⁷R⁸), (C₀₋₄-alkyl)-N(H)C(O)O(R⁷R⁸), (C₀₋₄-alkyl)-OR⁸, (C₀₋₄-alkyl)-imidazolyl, (C₀₋₄-alkyl)-pyrrolyl, (C₀₋₄-alkyl)-oxadiazolyl, or (C₀₋₄-alkyl)-triazolyl, or two of X₁, X₂, X₃, and X₄ may be joined together to form a cycloalkyl or heterocycloalkyl ring, (e.g. X₁ and X₂, X₂ and X₃, X₃ and X₄, X₁ and X₃, X₂ and X₄, or X₁ and X₄ may form a 3, 4, 5, 6, or 7 membered ring which may be aromatic, thereby forming a bicyclic system with the isoindolyl ring); and
R⁷ and R⁸ are each independently H, C₁₋₉-alkyl, C₃₋₆-cycloalkyl, (C₁₋₆-alkyl)-(C₃₋₆-cycloalkyl), (C₁₋₆-alkyl)-N(R⁷R⁸), (C₁₋₆-alkyl)-OR⁸, phenyl, benzyl, or aryl;
or a pharmaceutically acceptable salt, solvate, hydrate, stereoisomer, clathrate, or prodrug thereof.

Preferred compounds include, but are not limited to:
3-(4-Acetylamino-1,3-dioxo-1,3-dihydro-isoindol-2-yl)-3-(3-cyclopropylmethoxy-4-difluoromethoxy-phenyl)-propionic acid;
3-(4-Acetylamino-1,3-dioxo-1,3-dihydro-isoindol-2-yl)-3-(3-cyclopropylmethoxy-4-difluoromethoxy-phenyl)-N,N-dimethyl-propionamide;
3-(4-Acetylamino-1,3-dioxo-1,3-dihydro-isoindol-2-yl)-3-(3 cyclopropylmethoxy-4-difluoromethoxy-phenyl)-propionamide;
3-(3-Cyclopropylmethoxy-4-difluoromethoxy-phenyl)-3-(1,3-dioxo-1,3-dihydro-isoindol-2-yl)-propionic acid;
3-(3-Cyclopropylmethoxy-4-difluoromethoxy-phenyl)-3-(1,3-dioxo-1,3-dihydro-isoindol-2-yl)-N-hydroxy-propionamide;
3-(3-Cyclopropylmethoxy-4-difluoromethoxy-phenyl)-3-(7-nitro-1-oxo-1,3-dihydro-isoindol-2-yl)-propionic acid methyl ester;
3-(3-Cyclopropylmethoxy-4-difluoromethoxy-phenyl)-3-(7-nitro-1-oxo-1,3-dihydro-isoindol-2-yl)-propionic acid;
3-(3-Cyclopropylmethoxy-4-difluoromethoxy-phenyl-3-(7-nitro-1-oxo-1,3-dihydro-isoindol-2-yl)-)-N,N-dimethyl-propionamide;
3-(7-Amino-1-oxo-1,3-dihydro-isoindol-2-yl)-3-(3-cyclopropylmethoxy-4-difluoromethoxy-phenyl)-N,N-dimethyl-propionamide;
3-(4-Difluoromethoxy-3-ethoxy-phenyl)-3-(7-nitro-1-oxo-1,3-dihydro-isoindol-2-yl)-propionic acid methyl ester;
3-(7-Amino-1-oxo-1,3-dihydro-isoindol-2-yl)-3-(4-difluoromethoxy-3-ethoxy-phenyl)-propionic acid methyl ester;
3-[7-(Cyclopropanecarbonyl-amino)-1-oxo-1,3-dihydro-isoindol-2-yl]-3-(4-difluoromethoxy-3-ethoxy-phenyl)-propionic acid methyl ester;
3-(7-Acetylamino-1-oxo-1,3-dihydro-isoindol-2-yl)-3-(4-difluoromethoxy-3-ethoxy-phenyl)-propionic acid methyl ester;
3-(7-Acetylamino-1-oxo-1,3-dihydro-isoindol-2-yl)-3-(4-difluoromethoxy-3-ethoxy-phenyl)-propionic acid;
3-[7-(Cyclopropanecarbonyl-amino)-1-oxo-1,3-dihydro-isoindol-2-yl]-3-(4-difluoromethoxy-3-ethoxy-phenyl)-propionic acid;
Cyclopropanecarboxylic acid {2-[2-carbamoyl-1-(4-difluoromethoxy-3-ethoxy-phenyl)-ethyl]-3-oxo-2,3-dihydro-1H-isoindol-4-yl}-amide;
Cyclopropanecarboxylic acid {2-[1-(4-di fluoromethoxy-3-ethoxy-phenyl)-2-dimethylcarbamoyl-ethyl]-3-oxo-2,3-dihydro-1H-isoindol-4-yl}-;
Cyclopropanecarboxylic acid {2-[1-(4-difluoromethoxy-3-ethoxy-phenyl)-2-hydroxycarbamoyl-ethyl]-3-oxo-2,3-dihydro-1H-isoindol-4-yl}-amide;
3-(7-Acetylamino-1-oxo-1,3-dihydro-isoindol-2-yl)-3-(4-difluoromethoxy-3-ethoxy-phenyl)-propionamide;
3-(7-Acetylamino-1-oxo-1,3-dihydro-isoindol-2-yl)-3-(4-difluoromethoxy-3-ethoxy-phenyl)-N,N-dimethyl-propionamide;
3-(7-Acetylamino-1-oxo-1,3-dihydro-isoindol-2-yl)-3-(4-difluoromethoxy-3-ethoxy-phenyl)-N-hydroxy-propionamide;
3-(4-Acetylamino-1,3-dioxo-1,3-dihydro-isoindol-2-yl)-3-(4-difluoromethoxy-3-ethoxy-phenyl)-propionic acid;
3-(4-Acetylamino-1,3-dioxo-1,3-dihydro-isoindol-2-yl)-3-(4-difluoromethoxy-3-ethoxy-phenyl)-propionamide;
3-(4-Acetylamino-1,3-dioxo-1,3-dihydro-isoindol-2-yl)-3-(4-difluoromethoxy-3-ethoxy-phenyl)-N,N-dimethyl-propionamide;
3-(4-Acetylamino-1,3-dioxo-1,3-dihydro-isoindol-2-yl)-3-(4-difluoromethoxy-3-ethoxy-phenyl)-N-hydroxy-propionamide;
Cyclopropanecarboxylic acid {2-[1-(4-difluoromethoxy-3-ethoxy-phenyl)-2-methanesulfonyl-ethyl]-3-oxo-2,3-dihydro-1H-isoindol-4-yl}-amide;
N-{2-[1-(4-Difluoromethoxy-3-ethoxy-phenyl)-2-methanesulfonyl-ethyl]-1,3-dioxo-2,3-dihydro-1H-isoindol-4-yl}-acetamide; and
Cyclopropanecarboxylic acid {2-[2-carbamoyl-1-(4-difluoromethoxy-3-ethoxy-phenyl)-ethyl]-7-chloro-3-oxo-2,3-dihydro-1H-isoindol-4-yl}-amide.

Other selective cytokine inhibitory drugs include 7-amido-substituted isoindolyl compounds found in U.S. Provisional Application No. 60/454,155 to G. Muller et al., filed Mar. 12, 2003, which is incorporated herein in its entirety by reference. Representative 7-amido-substituted isoindolyl compounds include compounds of the formula: 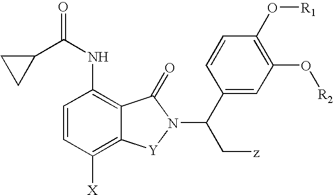
wherein:
Y is —C(O)—, —CH₂, —CH₂C(O)— or SO₂;
X is H,
Z is (C₀₋₄-alkyl)-C(O)R³, C₁₋₄-alkyl, (C₀₋₄-alkyl)-OH, (C₁₋₄-alkyl)-O(C₁₋₄-alkyl), (C₁₋₄-alkyl)-SO₂(C₁₋₄-alkyl), (C₀₋₄-alkyl)-SO(C₁₋₄-alkyl), (C₀₋₄-alkyl)-NH₂, (C₀₋₄-alkyl)-N(C₁₋₈-alkyl)₂, (C₀₋₄-alkyl)-N(H)(OH), CH₂NSO₂(C₁₋₄-alkyl);
   R₁ and R₂ are independently C₁₋₈-alkyl, cycloalkyl, or (C₁₋₄-alkyl)cycloalkyl;
   R³ is, NR⁴R⁵, OH, or O—(C₁₋₈-alkyl);
   R⁴ is H;
   R⁵ is —OH, or —OC(O)R⁶;
   R⁶ is C₁₋₈-alkyl, amino-(C₁₋₈-alkyl), (C₁₋₈-alkyl)-(C₃₋₆-cycloalkyl), C₃₋₆cycloalkyl, phenyl, benzyl, or aryl;
   or a pharmaceutically acceptable salt, solvate, hydrate, stereoisomer, clathrate, or prodrug thereof; or the formula: 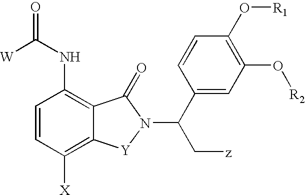
   wherein:
   Y is —C(O)—, —CH₂, —CH₂C(O)—, or SO₂;
   X is halogen, —CN, —NR₇R₈, —NO₂, or —CF₃,
   W is 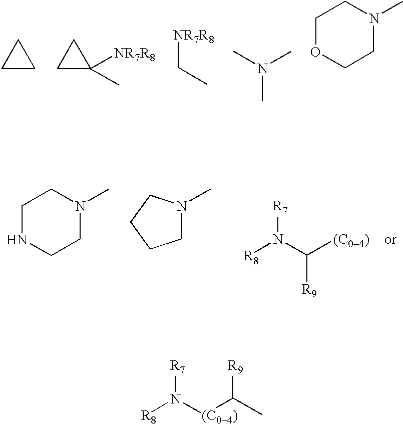
   Z is (C₀₋₄alkyl)-SO₂(C₁₋₄-alkyl), —(C₀₋₄alkyl)-CN, —(C₀₋₄alkyl)-C(O)R³, C₁₋₄-alkyl, (C₀₋₄-alkyl)OH, (C₀₋₄-alkyl)O(C₁₋₄-alkyl), (C₀₋₄-alkyl)SO(C₁₋₄-alkyl), (C₀₋₄-alkyl)NH₂, (C₀₋₄-alkyl)N(C₁₋₈-alkyl)₂, (C₀₋₄-alkyl) N(H)(OH), or (C₀₋₄-alkyl)NSO₂(C1-4-alkyl);
   W is —C₃₋₆-cycloalkyl, —(C₁₋₈-alkyl)-(C₃₋₆-cycloalkyl), —(C₀₋₈-alkyl)-(C₃₋₆cycloalkyl)-NR₇R₈, (C₀₋₈-alkyl)-NR₇R₈, (C₀₋₄-alkyl)-CHR₉—(C₀₋₄-alkyl)-NR₇R₈,
   R₁ and R₂ are independently C₁₋₈-alkyl, cycloalkyl, or (C₁₋₄-alkyl)cycloalkyl;
   R³ is C₁₋₈-alkyl, NR⁴R⁵, OH, or O—(C₁₋₈-alkyl);
   R⁴ and R⁵ are independently H, C₁₋₈-alkyl, (C₀₋₈-alkyl)-(C₃₋₆-cycloalkyl), OH, or —OC(O)R⁶
   R⁶ is C₁₋₈-alkyl, (C₀₋₈-alkyl)-(C₃₋₆-cycloalkyl), amino-(C₁₋₈-alkyl), phenyl, benzyl, or aryl;
   R₇ and R₈ are each independently H, C₁₋₈-alkyl, (C₀₋₈alkyl)-(C₃₋₆-cycloalkyl), phenyl, benzyl, aryl, or can be taken together with the atom connecting them to form a 3 to 7 membered heterocycloalkyl or heteroaryl ring;
   R₉ is C₁₋₄-alkyl, (C₀₋₄-alkyl)aryl, (C₀₋₄-alkyl)-(C₃₋₆-cycloalkyl), (C₀₋₄-alkyl)-heterocylcle;
   or a pharmaceutically acceptable salt, solvate, hydrate, stereoisomer, clathrate, or prodrug thereof.

Still other selective cytokine inhibitory drugs include N-alkyl-hydroxamic acid-isoindolyl compounds found in U.S. Provisional Application No. 60/454,149 to G. Muller et al., filed Mar. 12, 2003, which is incorporated herein in its entirety by reference. Representative N-alkyl-hydroxamic acid-isoindolyl compounds include compounds of the formula: 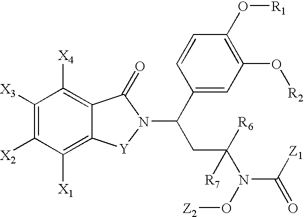
wherein:
Y is —C(O)—, —CH₂, —CH₂C(O)— or SO₂;
R₁ and R₂ are independently C₁₋₈-alkyl, CF₂H, CF₃, CH₂CHF₂, cycloalkyl, or (C₁₋₈-alkyl)cycloalkyl;
Z₁ is H, C₁₋₆-alkyl, —NH₂—NR₃R₄ or OR₅;
Z₂ is H or C(O)R₅;
X₁, X₂, X₃ and X₄ are each independent H, halogen, NO₂, OR₃, CF₃, C₁₋₆-alkyl, (C₀₋₄-alkyl)-(C₃₋₆-cycloalkyl), (C₀₋₄-alkyl)-N—(R₈R₉), (C₀₋₄-alkyl)-NHC(O)—(R₈), (C₀₋₄-alkyl)-NHC(O)CH(R₉)(R₉), (C₀₋₄-alkyl)-NHC(O)N(R₈R₉), (C₀₋₄-alkyl)-NHC(O)O(R₈), (C₀₋₄-alkyl)-O—R₈, (C₀₋₄-alkyl)-imidazolyl, (C₀₋₄-alkyl)-pyrrolyl, (C₀₋₄-alkyl)-oxadiazolyl, (C₀₋₄-alkyl)-triazolyl or (C₀₋₄-alkyl)-heterocycle;
R₃, R₄, and R₅ are each independently H, C₁₋₆-alkyl, O—C₁₋₆-alkyl, phenyl, benzyl, or aryl;
R₆ and R₇ are independently H or C₁₋₆-alkyl;
R₈ and R₉ are each independently H, C₁₋₉-alkyl, C₃₋₆-cycloalkyl, (C₁₋₆-alkyl)-(C₃₋₆-cycloalkyl), (C₀₋₆-alkyl)-N(R₄R₅), (C₁₋₆-alkyl)-OR₅, phenyl, benzyl, aryl, piperidinyl, piperizinyl, pyrolidinyl, morpholino, or C₃₋₇-heterocycloalkyl; and
or a pharmaceutically acceptable salt, solvate, hydrate, stereoisomer, clathrate, or prodrug thereof.

Specific selective cytokine inhibitory drugs include, but are not limited to:
2-[1(-3-ethoxy-4-methoxyphenyl)-2-methyl-sulfonylethyl]isoindolin-1-one;
2-[1-(3-ethoxy-4-methoxyphenyl)-2-(N,N-dimethyl-aminosulfonyl)ethyl]isoindolin-1-one;
2-[1-(3-ethoxy-4-methoxyphenyl)-2-methyl-sulfonylethyl]isoindoline-1,3-dione;
2-[1-(3-ethoxy-4-methoxyphenyl)-2-methyl-sulfonylethyl]-5-nitro-isoindoline-1,3-dione;
2-[1-(3-ethoxy-4-methoxyphenyl)-2-methyl-sulfonylethyl]-4-nitroisoindoline-1,3-dione;
2-[1-(3-ethoxy-4-methoxyphenyl)-2-methylsulfonylethyl]-4-aminoisoindoline-1,3-dione;
2-[1-(3-ethoxy-4-methoxyphenyl)-2-methylsulfonylethyl]-5-methylisoindoline-1,3-dione;
2-[1-(3-ethoxy-4-methoxyphenyl)-2-methylsulfonylethyl]-5-acetamidoisoindoline-1,3-dione;
2-[1-(3-ethoxy-4-methoxyphenyl)-2-methylsulfonylethyl]-4-dimethylaminoisondoline-1,3-dione;
2-[1-(3-ethoxy-4-methoxyphenyl)-2-methylsulfonylethyl]-5-dimethylaminoisoindoline-1,3-dione;
2-[1-(3-ethoxy-4-methoxyphenyl)-2-methylsulfonylethyl]benzo[e]isoindoline-1,3-dione;
2-[1-(3-ethoxy-4-methoxyphenyl)-2-methylsulfonylethyl]-4-methoxyisoindoline-1,3-dione;
1-(3-cyclopentyloxy-4-methoxyphenyl)-2-methylsulfonylethyl-amine;
2-[1-(3-cyclopentyloxy-4-methoxyphenyl)-2-methylsulfonylethyl]isoindoline-1,3-dione; and
2-[1-(3-cyclopentyloxy-4-methoxyphenyl)-2-methylsulfonylethyl]-4-dimethylaminoisoindoline-1,3-dione.

Additional selective cytokine inhibitory drugs include the enantiomerically pure compounds disclosed in U.S. provisional patent application Nos. 60/366,515 and 60/366,516 to G. Muller et al., both of which were filed Mar. 20, 2002, and U.S. provisional patent application Nos. 60/438,450 and 60/438,448 to G. Muller et al., both of which were filed on Jan. 7, 2003, and all of which are incorporated herein by reference. Preferred compounds include an enantiomer of 2-[1-(3-ethoxy-4-methoxyphenyl)-2-methylsulfonylethyl]-4-acetylaminoisoindoline-1,3-dione and an enantiomer of 3-(3,4-dimethoxy-phenyl)-3-(1-oxo-1,3-dihydro-isoindol-2-yl)-propionamide.

Preferred selective cytokine inhibitory drugs used in the invention are 3-(3,4-dimethoxy-phenyl)-3-(1-oxo-1,3-dihydro-isoindol-2-yl)-propionamide and cyclopropanecarboxylic acid {2-[1-(3-ethoxy-4-methoxy-phenyl)-2-methanesulfonyl-ethyl]-3-oxo-2,3-dihydro-1H-isoindol-4-yl}-amide, which are available from Celgene Corp., Warren, N.J. 3-(3,4-dimethoxy-phenyl)-3-(1-oxo-1,3-dihydro-isoindol-2-yl)-propionamide has the following chemical structure:

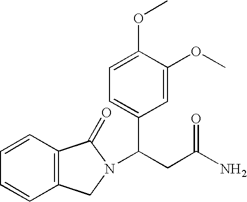

Cyclopropanecarboxylic acid {2-[1-(3-ethoxy-4-methoxy-phenyl)-2-methanesulfonyl-ethyl]-3-oxo-2,3-dihydro-1H-isoindol-4-yl}-amide has the following chemical structure:

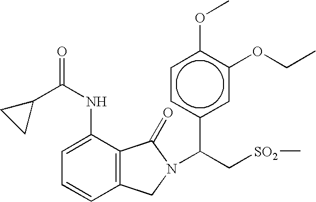

The compounds of the invention also include, but are not limited to, compounds that inhibit PDE IV activity, such as cilomast, theophylline, zardaverine, rolipram, pentoxyfylline, enoximone, isoindole-imides, phenethylsulfones, alkanohydroxamic acids, non-polypeptide cyclic amides, oxoisoindoles, isoindolines, indazoles, heterosubstituted pyridines, diphenylpyridines, aryl thiophenes, aryl furans, indenes, trisubstituted phenyls, phthalazinones, benzenesulfonamides, tetracyclic compounds and salts, solvates, isomers, clathrates, pro-drugs, hydrates or derivatives thereof. In one embodiment, the compound is not a polypeptide, peptide, protein, hormone, cytokine, oligonucleotide or nucleic acid.

In another embodiment, the compounds of this invention have the following structure (I): 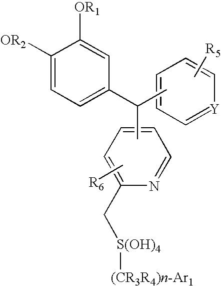
including isomers, prodrugs and pharmaceutically acceptable salts, hydrates, solvates, clathrates thereof, wherein:
Y represents N or N-oxide;
R₁ and R₂ are independently selected from:
H, C₁₋₆ alkyl and halo C₁₋₆ alkyl;
R₃ and R₄ are independently selected from H and C₁₋₆ alkyl, or R₃ and R₄ attached to the same carbon atom taken together represent a carbonyl oxygen atom, or R₃ and R₄ attached to different carbon atoms considered in combination with the carbon atoms to which they are attached along with any intervening atoms and represent a saturated 5, 6 or 7 membered carbocyclic ring;
R₅ and R₆ independently represent a member selected from the group consisting of: H, C₁₋₆ alkyl, halo C₁₋₆ alkyl and CN;
n represents an integer of from 0-6;
Ar₁ is selected from the group consisting of:
thienyl, thiazolyl, pyridyl, phenyl and naphthyl; said Ar₁ being optionally substituted with 1-3 members selected from the group consisting of: halo, C₁₋₆ alkoxy, C₁₋₇ alkylthio, CN,
C₁₋₆ alkyl, hydroxy C₁₋₆ alkyl, —C(O)C₁₋₆ alkyl, —CO₂H, —CO₂C₁₋₆ alkyl, NH(SO₂Me), N(SO₂Me)₂, SO₂Me, SO₂ NH₂, SO₂NHC₁₋₆ alkyl, SO₂N(C₁₋₆ alkyl)₂ NO₂, C₂₋₆ alkenyl,
C₁₋₆ alkyl, and NH₂;
and when Ar₁ represents a phenyl or naphthyl group with two or three substituents, two such substituents may be considered in combination and represent a 5 or 6 membered fused lactone ring.

This embodiment further encompasses compounds such as those found in U.S. Pat. No. 6,316,472, which is incorporated herein by reference in its entirety.

In another embodiment, the compounds of the invention have the following structure (II): 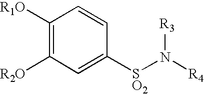
including isomers, prodrugs and pharmaceutically acceptable salts, hydrates, solvates, clathrates thereof, wherein:
R₁ and R₂ represent C₁-C₄ alkyl or C₃-C₁₀ cycloalkyl;
R₃ and R₄ independently represent C₁₋₄ alkyl, cycloalkyl, C₂-C₄ alkylenes having one double bond, C₂-C₄ alkylynes having one triple bond, (CH₂)ₙCO(CH₂)ₘCH₃, (CH₂)ₚCN, (CH₂)ₚCO₂Me, or taken together with nitrogen atom to which they are attached, form a 3- to 10-membered ring;
n and m are 0 to 3;
p is 1 to 3.

This embodiment further encompasses compounds such as those found in U.S. Pat. No. 6,162,830, which is incorporated herein by reference in its entirety.

In another embodiment, the compounds of this invention have the following structure (III): 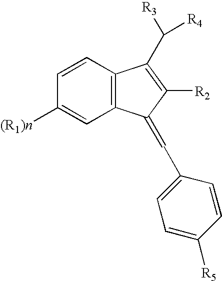
including isomers, prodrugs and pharmaceutically acceptable salts, hydrates, solvates, clathrates thereof, wherein:
R₁ is independently selected in each instance from the group consisting of hydrogen, halogen, lower alkoxy, hydroxy, lower alkyl, lower alkyl mercapto, lower alkylsulfonyl, lower alkylamino, di-lower alkyl amino, amino, nitro, nitrile, lower alkyl carboxylate, —CO₂H, and sulfonamido;
R₂ is selected from the group consisting of hydrogen and lower alkyl;
R₃ is selected from the group consisting of hydrogen, lower alkyl, hydroxy, and amino;
R₄ is selected from the group consisting of —COM and CH₂OH wherein M is selected from the group consisting of:
hydroxy, substituted lower alkoxy, amino, alkylamino, dialkylamino, N-morpholino, hydroxyalkylamino, polyhydroxyamino, dialkylaminoalkylamino, aminoalklyamino, and the group OMe, wherein Me is a cation;
R₅ is an alkyl sulfonyl; and
n is an integer from 0 to four.

This embodiment further encompasses compounds disclosed in U.S. Pat. No. 6,177,471, which is incorporated herein by reference in its entirety.

In another embodiment, the compounds of this invention have the following structure (IV): 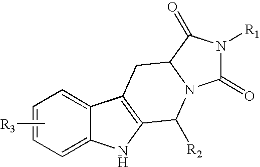
including isomers, prodrugs and pharmaceutically acceptable salts, hydrates, solvates, clathrates thereof, wherein:
R₀ represents hydrogen, halogen, or C₁₋₆ alkyl;
R₁ is selected from the group consisting of:
hydrogen; C₁₋₆ alkyl optionally substituted by one or more substituents selected from phenyl, halogen, —CO₂Rₐ, —NRₐR_{b}, C₃₋₆-cycloalkyl, phenyl, and a 5- or 6-membered heterocyclic ring selected from the group consisting of pyridyl, morpholinyl, piperazinyl, pyrrolidinyl, and piperidinyl, and being optionally substituted by one or more C₁₋₆ alkyl, and optionally linked to the nitrogen atom to which R₁ is attached via C₁₋₆ alkyl;
R₂ is selected from the group consisting of:
phenyl optionally substituted by one or more substituents selected from —ORₐ, —NRₐ, R_{b}, halogen, hydroxy, trifluoromethyl, cyano, and nitro;
and Rₐ and R_{b} independently represent hydrogen or C₁₋₆ alkyl including isomers, prodrugs and pharmaceutically acceptable salts thereof.

This embodiment further encompasses compounds such as those found in U.S. Pat. No. 6,218,400, which is incorporated herein by reference in its entirety.

In another embodiment, the compounds of this invention have the following structure (V): 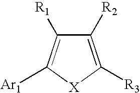
including isomers, prodrugs and pharmaceutically acceptable salts, hydrates, solvates, clathrates thereof, wherein:
X is S or O;
Ar₁ is an aromatic ring selected from phenyl, pyridinyl, or furyl, optionally substituted with up to two substituents, each substituent independently is:
C₁₋₆ alkyl, optionally substituted with —OH, —CO₂H, CO₂C₁₋₃ alkyl, or CN; C₁₋₆ alkoxy; C₁₋₃ alkylthio, C₁₋₃ alkylsulfonyl, C₁₋₃ fluoroalkyl, optionally substituted with —OH; halo, —OH, —CO₂H, or —CO₂ C₁₋₃ alkyl;
R₂ is hydrogen or C₁₋₃ alkyl; and
R₃ is phenyl, pyridinyl, quinolinyl or furyl, optionally substituted with up to two substituents, each substituent independently is: C₁₋₃ alkyl, C₁₋₃ fluoroalkyl, C₁₋₆ alkoxy, C₁₋₃ fluoroalkoxy, C₁₋₃ alkylthio, halo, or —OH.

This embodiment further encompasses compounds such as those found in U.S. Pat. No. 6,034,089 and U.S. Pat. No. 6,020,339, which are incorporated herein by reference in their entireties.

In another embodiment, the compounds of this invention have the following structure (VI): 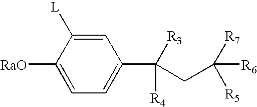
including isomers, prodrugs and pharmaceutically acceptable salts, hydrates, solvates, clathrates thereof, wherein:
Y is halogen or an alkyl or —XRₐ group;
Z is —O—, —S(O)ₚ— or —N(R_{b})—, where p is zero or an integer 1 or 2;
L is —XR, —C(R₁₁)C(R₁)(R₂) or —(CHR₁₁)ₙCH(R₁)(R₂), where n is zero or the integer 1;
each of Rₐ and R_{b} is independently hydrogen or an optionally substituted alkyl group;
R is an optionally substituted alkyl, alkenyl, cycloalkyl or cycloalkenyl group;
each of R₁ and R₂, which may be the same or different, is hydrogen, fluorine, —CN, —NO₂, or an optionally substituted alkyl, alkenyl, alkynyl, alkoxy, alkylthio, CO₂R₈, —CONR₉R₁₀ or —CSNR₉R₁₀ group, or R₁ and R₂, together with the carbon atom to which they are attached, are linked to form an optionally substituted cycloalkyl or cycloalkenyl group;
R₃ is hydrogen, fluorine, hydroxy or an optionally substituted straight or branched alkyl group;
R₄ is hydrogen, —(CH₂)ₜAr or —(CH₂)ₜ—Ar-(L₁)ₙ—Ar₁, where t is zero or an integer 1, 2 or 3;
R₅ is —(CH₂)ₜAr or —(CH₂)ₜ—Ar-(L₁)ₙ—Ar′;
R₆ is hydrogen, fluorine, or an optionally substituted alkyl group;
R₇ is hydrogen, fluorine, an optionally substituted straight or branched alkyl group, —ORc, where Rc is hydrogen or an optionally substituted alkyl or alkenyl group, or a formyl, alkoxyalkyl, alkanoyl, carboxamido or thiocarboxamido group;
each of R₈, R₉ and R₁₀ is independently hydrogen or an optionally substituted alkyl, aralkyl or aryl group; and
R₁₁ is hydrogen, fluorine or a methyl group.

This embodiment further encompasses compounds such as those found in U.S. Pat. No. 5,798,373, which is incorporated herein by reference in its entirety.

In a preferred embodiment, the compound is of structure (VII): 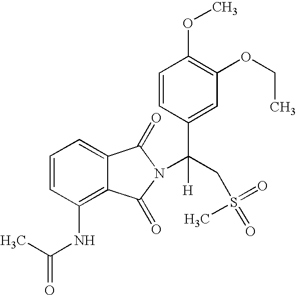
or a pharmaceutically acceptable salt, hydrate, solvate, clathrate, enantiomer, diastereomer, racemate, or mixture of stereoisomers thereof.

In another preferred embodiment, the compound is that of structure (VIII): 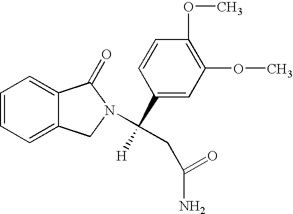
including isomers, salts, clathrates, solvates, hydrates, prodrugs and pharmaceutically acceptable salts thereof.

Certain of these compounds may be commercially available from Celgene, Inc., Warren, N.J. Other above compounds can be made by methods known in the art, including those disclosed in the patents cited above which are incorporated by reference in their entireties.

Additional examples of PDE IV inhibitors which are useful in the methods of the present invention include those disclosed in GB 2 063 249 A, EP 0 607 439 A1, U.S. Pat. No. 6,333,354, U.S. Pat. No. 6,300,335, U.S. Pat. No. 6,166,041, U.S. Pat. No. 6,069,156, U.S. Pat. No. 6,011,060, U.S. Pat. No. 5,891,896, U.S. Pat. No. 5,849,770, U.S. Pat. No. 5,710,170, U.S. Pat. No. 4,101,548, U.S. Pat. No. 4,001,238, U.S. Pat. No. 4,001,237, U.S. Pat. No. 3,920,636, U.S. Pat. No. 4,060,615, WO 97/03985, EP 0 607 439 A1, U.S. Pat. No. 4,101,548, U.S. Pat. No. 4,001,238, U.S. Pat. No. 4,001,237, U.S. Pat. No. 3,920,636, U.S. Pat. No. 4,060,615, WO 97/03985, EP 0 395 328, U.S. Pat. No. 4,209,623, EP 0 395 328, U.S. Pat. No. 4,209,623, U.S. Pat. No. 5,354,571, EP 0 428 268 A2, U.S. Pat. No. 5,354,571, EP 0 428 268 A2, 807,826, U.S. Pat. No. 3,031,450, U.S. Pat. No. 3,322,755, U.S. Pat. Nos. 5,401,774, 807,826, U.S. Pat. No. 3,031,450, U.S. Pat. No. 3,322,755, U.S. Pat. No. 5,401,774, U.S. Pat. No. 5,147,875, PCT WO 93/12095, U.S. Pat. No. 5,147,875, PCT WO 93/12095, U.S. Pat. No. 4,885,301, WO 93/07149, EP 0 349 239 A2, EP 0 352 960 A2, EP 0 526 004 A1, EP 0 463 756 A1, U.S. Pat. No. 4,885,301, WO 93/07149, EP 0 349 239 A2, EP 0352 960 A2, EP 0 526 004 A1, EP 0 463 756 A1, EP 0 607 439 A1, EP 0 607 439 A1, WO 94/05661, EP 0 351 058, U.S. Pat. No. 4,162,316, EP 0 347 146, U.S. Pat. No. 4,047,404, U.S. Pat. No. 5,614,530, U.S. Pat. No. 5,488,055, WO 97/03985, WO 97/03675, WO 95/19978, U.S. Pat. No. 4,880,810, WO 98/08848, U.S. Pat. No. 5,439,895, U.S. Pat. No. 5,614,627, PCT US94/01728, WO 98/16521, EP 0 722 943 A1, EP 0 722 937 A1, EP 0 722 944 A1, WO 98/17668, WO 97/24334, WO 97/24334, WO 97/24334, WO 97/24334, WO 97/24334, WO 98/06722, PCT/JP97/03592, WO 98/23597, WO 94/29277, WO 98/14448, WO 97/03070, WO 98/38168, WO 96/32379 and PCT/GB98/03712, all of which are incorporated herein by reference.

Many of the compounds that are contemplated as part of the present invention can be enriched in optically active enantiomers of the compounds specified above using standard resolution or asymmetric synthesis known in the art. See, e.g., Shealy et al., Chem. Indus. 1030 (1965); and Casini et al., Farmaco Ed. Sci. 19: 563 (1964).

The present invention also pertains to the physiologically acceptable non-toxic acid addition salts of the compounds thereof. Such salts include those derived from organic and inorganic acids or bases know in the art: such acids include for example, hydrochloric acid, hydrobromic acid, phosphoric acid, sulfinuric acid, methanesulphonic acid, acetic acid, tartaric acid, lactic acid, succinic acid, citric acid, malic acid, maleic acid, sorbic acid, aconitic acid, salicylic acid, phthalic acid, embolic acid, enanthic acid, and the like.

Compounds of the invention that are acidic in nature are capable of forming salts with various pharmaceutically acceptable bases. The bases that can be used to prepare pharmaceutically acceptable base addition salts of such acidic compounds of the invention are those that form non-toxic base addition salts, ie., salts containing pharmacologically acceptable cations such as, but not limited to, alkali metal or alkaline earth metal salts and the calcium, magnesium, sodium or potassium salts in particular. Suitable organic bases include, but are not limited to, N,N-dibenzylethylenediamine, chloroprocaine, choline, diethanolamine, ethylenediamine, meglumaine (N-methylglucamine), lysine, and procaine.

The compounds of the invention can be assayed for their ability to inhibit PDE IV using methods well known in the art, for example, those assays disclosed in U.S. Pat. No. 6,316,472; U.S. Pat. No. 6,204,275; Featherstone R. L. et al. (2000) “Comparison of phosphodiesterase inhibitors of differing isoenzyme selectivity added to St. Thomas' hospital cardioplegic solution used for hypothermic preservation of rat lungs”, Am. J. Respir Crit. Care Med. 162: 850-6; and Brackeen M. F. et al. (1995) “Design and synthesis of conformationally constrained analogues of 4-(3-butoxy-4-methoxybenzyl)imidazolidin-2-one (Ro 20-1724) as potent inhibitors of cAMP-specific phosphodiesterase”, J. Med. Chem. 38: 4848-54, which are incorporated herein by reference in their entirety.

The compounds of the invention can either be commercially purchased from Celgene Corp. (Warren, N.J.), or may be prepared according to the methods described in the patents or patent publications disclosed herein. Further, optically pure compositions can be asymmetrically synthesized or resolved using known resolving agents or chiral columns as well as other standard synthetic organic chemistry techniques.

5.3 Stem Cell Populations

The present invention provides methods of identifying compounds that modulate human angiogenesis. Any human stem cell can be used within the methods of the invention, including, but not limited to, stem cells isolated from cord blood (CB cells), peripheral blood, adult blood, bone marrow, placenta, mesenchymal stem cells and other sources. In a non-preferred embodiment, the stem cells are embryonic stem cells that have been isolated from sources other than placenta.

Sources of mesenchymal stem cells include bone marrow, embryonic yolk sac, placenta, umbilical cord, fetal and adolescent skin, and blood. Bone marrow cells may be obtained from iliac crest, femora, tibiae, spine, rib or other medullary spaces.

The stem cells to be used in accordance with the methods of the present invention may include pluripotent cells, i.e., cells that have complete differentiation versatility, that are self-renewing, and can remain dormant or quiescent within tissue. The stem cells may also include multipotent cells, committed progenitor cells, and fibroblastoid cells. In one preferred embodiment, the invention utilizes stem cells that are viable, quiescent, pluripotent stem cells isolated from a full-term exsanguinated perfused placenta.

Stem cell populations may consist of placental stem cells obtained through a commercial service, e.g., LifeBank USA (Cedar Knolls, N.J.), ViaCord (Boston Mass.), Cord Blood Registry (San Bruno, Calif.) and Cryocell (Clearwater, Fla.).

Stem cell populations may also consist of placental stem cells collected according to the methods disclosed in U.S. Application Publication No. U.S. 2002/0123141, published Sep. 5, 2002, entitled “Method of Collecting Placental Stem Cells” and U.S. Application Publication No. U.S. 2003/0032179, published Feb. 13, 2003, entitled “Post-Partum Mammalian Placenta, Its Use and Placental Stem Cells Therefrom” (both of which are incorporated herein by reference in their entireties).

Preferred cells to be used in accordance with the present invention are embryonic-like stem cells that originate from an exsanguinated perfused placenta, or cells that derive from embryonic-like placental stem cells. The embryonic-like stem cells of the invention may be characterized by measuring changes in morphology and cell surface markers using techniques such as flow cytometry and immunocytochemistry, and measuring changes in gene expression using techniques, such as PCR. In one embodiment of the invention, such embryonic-like stem cells may be characterized by the presence of the following cell surface markers: CD10, CD29, CD44, CD54, CD90, SH2, SH3, SH4, OCT-4 and ABC-p, or the absence of the following cell surface markers: CD34, CD38, CD45, SSEA3 and SSEA4. In a preferred embodiment, such embryonic-like stem cells may be characterized by the presence of cell surface markers OCT-4+ and APC-p+. Such cell surface markers are routinely determined according to methods well known in the art, e.g. by flow cytometry, followed by washing and staining with an anti-cell surface marker antibody. For example, to determine the presence of CD-34 or CD-38, cells may be washed in PBS and then double-stained with anti-CD34 phycoerythrin and anti-CD38 fluorescein isothiocyanate (Becton Dickinson, Mountain View, Calif.).

Embryonic-like stem cells originating from placenta have characteristics of embryonic stem cells but are not derived from the embryo. In other words, the invention encompasses the use of OCT-4+ and ABC-p+ cells that are undifferentiated stem cells that are isolated from a postpartum perfused placenta. Such cells are as versatile (e.g., pluripotent) as human embryonic stem cells. As mentioned above, a number of different pluripotent or multipotent stem cells can be isolated from the perfused placenta at different time points e.g., CD34+/CD38+, CD34+/CD38−, and CD34−/CD38− hematopoietic cells. According to the methods of the invention, human placenta is used post-birth as the source of embryonic-like stem cells.

For example, after expulsion from the womb, the placenta is exsanguinated as quickly as possible to prevent or minimize apoptosis. Subsequently, as soon as possible after exsanguination the placenta is perfused to remove blood, residual cells, proteins, factors and any other materials present in the organ. Materials debris may also be removed from the placenta. Perfusion is normally continued with an appropriate perfusate for at least two to more than twenty-four hours. In several additional embodiments the placenta is perfused for at least 4, 6, 8, 10, 12, 14, 16, 18, 20, and 22 hours. In other words, this invention is based at least in part on the discovery that the cells of a postpartum placenta can be activated by exsanguination and perfusion for a sufficient amount of time. Therefore, the placenta can readily be used as a rich and abundant source of embryonic-like stem cells, which cells can be used for research, including drug discovery, treatment and prevention of diseases, in particular transplantation surgeries or therapies, and the generation of committed cells, tissues and organoids. See, U.S. Application Publication No. U.S. 20020123141, published Sep. 5, 2002, entitled “Method of Collecting Placental Stem Cells” and U.S. Application Publication No. U.S. 2003/0032179, published Feb. 13, 2003, entitled “Post-Partum Mammalian Placenta, Its Use and Placental Stem Cells Therefrom” (both of which are incorporated herein by reference in their entireties).

Embryonic-like stem cells are extracted from a drained placenta by means of a perfusion technique that utilizes either or both of the umbilical artery and umbilical vein. The placenta is preferably drained by exsanguination and collection of residual blood (e.g., residual umbilical cord blood). The drained placenta is then processed in such a manner as to establish an ex vivo, natural bioreactor environment in which resident embryonic-like stem cells within the parenchyma and extravascular space are recruited. The embryonic-like stem cells migrate into the drained, empty microcirculation where, according to the methods of the invention, they are collected, preferably by washing into a collecting vessel by perfusion.

5.4 Methods of Stem Cell Culture

In certain embodiments of the invention, stem or progenitor cells, including but not limited to embryonic stem cells, embryonic-like stem cells, progenitor cells, pluripotent cells, totipotent cells, multipotent cells, cells endogenous to a postpartum perfused placenta, cord blood cells, stem or progenitor cells derived from peripheral blood or adult blood, or bone marrow cells, are used in the in vitro screening assays of the present invention.

In another embodiment of the invention, the stem or progenitor cells are not derived from a postpartum perfused placenta but instead, are isolated from other sources such as cord blood, bone marrow, peripheral blood or adult blood, are exposed to the compounds of the invention and assayed for angiogenesis.

In another embodiment, the cultured stem cells, e.g., stem cells cultured in vitro or in a postpartum perfused placenta, are stimulated to proliferate in culture, for example, by administration of erythropoietin, cytokines, lymphokines, interferons, colony stimulating factors (CSF's), interferons, chemokines, interleukins, recombinant human hematopoietic growth factors including ligands, stem cell factors, thrombopoeitin (Tpo), interleukins, and granulocyte colony-stimulating factor (G-CSF) or other growth factors.

5.4.1 Stem Cell Culture In Vitro

Methods for culturing stem or progenitor cells in vitro are well known in the art, e.g., see, Thomson et al., 1998, Science 282: 1145-47 (embryonic stem cells); Hirashima et al., 1999, Blood 93(4): 1253-63, and Hatzopoulos et al., 1998, Development 125: 1457-1468 (endothelial cell progenitors); Slager et al, 1993, Dev. Genet. 14(3): 212-24 (neuron or muscle progenitors); Genbachev et al., 1995, Reprod. Toxicol. 9(3): 245-55 (cytotrophoblasts, i.e., placental epithelial cell progenitors); Nadkarni et al. 1984, Tumori 70: 503-505, Melchner et al., 1985, Blood 66(6): 1469-1472, international PCT publication WO 00/27999 published May 18, 2000, Himori et al., 1984, Intl. J. Cell Cloning 2: 254-262, and Douay et al., 1995, Bone Marrow Transplantation 15: 769-775 (hematopoietic progenitor cells); Shamblott et al., 1998, Proc. Natl. Acad. Sci. USA 95: 13726-31 (primordial germ cells); Yan et al., 2001, Devel. Biol. 235: 422-432 (trophoblast stem cells).

5.4.2 Stem Cell Culture in a Postpartum Perfused Placenta

The methods of the present invention encompass the use of pluripotent stem cells derived from a placenta. Methods of obtaining and culturing such cells, as described below, is described in detail in U.S. Application Publication No. U.S. 20020123141, published Sep. 5, 2002, entitled “Method of Collecting Placental Stem Cells” and U.S. Application Publication No. U.S. 20030032179, published Feb. 13, 2003, entitled “Post-Partum Mammalian Placenta, Its Use and Placental Stem Cells Therefrom,” both of which are incorporated herein by reference in their entireties.

5.4.2.1 Pretreatment of Placenta

According to the methods of the invention, a human placenta is recovered shortly after its expulsion after birth and, in certain embodiments, the cord blood in the placenta is recovered. In certain embodiments, the placenta is subjected to a conventional cord blood recovery process. A needle or cannula is typically used, with the aid of gravity, to drain cord blood from (i.e., exsanguinate) the placenta (Boyse et al., U.S. Pat. No. 5,192,553, issued Mar. 9, 1993; Boyse et al., U.S. Pat. No. 5,004,681, issued Apr. 2, 1991; Anderson, U.S. Pat. No. 5,372,581, issued Dec. 13, 1994; Hessel et al., U.S. Pat. No. 5,415,665, entitled Umbilical cord clamping, cutting, and blood collecting device and method, issued May 16, 1995). Such cord blood recovery may be obtained commercially, e.g., LifeBank. USA (Cedar Knolls, N.J.), ViaCord (Boston Mass.), Cord Blood Registry (San Bruno, Calif.) and Cryocell (Clearwater, Fla.). The cord blood can be drained shortly after expulsion of the placenta.

Postpartum the placenta is drained of cord blood. The placenta stored nay be under sterile conditions and at either room temperature or at a temperature of 5 to 25° C. (centigrade). The placenta may be stored for a period of longer than forty eight hours, and preferably for a period of four to twenty-four hours prior to perfusing the placenta to remove any residual cord blood.

The placenta is preferably recovered after expulsion under aseptic conditions, and stored in an anticoagulant solution at a temperature of 5 to 25° C. (centigrade). Suitable anticoagulant solutions are well known in the art. For example, a solution of heparin or warfarin sodium can be used, e.g., a solution of heparin (1% w/w in 1:1000 solution). The drained placenta is preferably stored for no more than 36 hours before the embryonic-like stem cells are collected. The solution that is used to perfuse the placenta to remove residual cells can be the same solution used to perfuse and culture the placenta for the recovery of stem cells. Any of these perfusates may be collected and used as a source of embryonic-like stem cells.

The placenta may also be recovered from a patient by informed consent and a complete medical history of the patient prior to, during and after pregnancy is also taken: and is associated with the placenta. These medical records can be used to coordinate subsequent use of the placenta or the stem cells harvested therefrom. For example, the human placental stem cells can then easily be used for personalized medicine for the infant in question, the parents, siblings or other relatives. Indeed, the human placental stem cells are more versatile than cord blood. However, it should be noted that the invention includes the addition of human placental stem cells produced by the exsanguinated, perfused and/or cultured placenta to cord, blood from the same or different placenta and umbilical cord. The resulting cord blood will have an increased concentration/population of human stem cells and thereby is more useful for transplantation e.g. for bone marrow transplantations.

5.4.2.2 Exsanguination of Placenta and Removal of Residual Cells

According to certain embodiments of the invention, stem or progenitor cells, including, but not limited to embryonic-like stem cells, may be recovered from a placenta that is exsanguinated, i.e., completely drained of the cord blood remaining afterbirth and/or a conventional cord blood recovery procedure.

5.4.2.3 Culture of Placenta and Stem Cells Therein

After exsanguination and a sufficient time of perfusion of the placenta; the embryonic-like stem cells are observed to migrate into the exsanguinated and perfused microcirculation of the placenta where, according to the methods of the invention, they are collected, preferably by washing into a collecting vessel by perfusion. Perfusing the isolated placenta not only serves to remove residual cord blood but also provide the placenta with the appropriate nutrients, including oxygen. The placenta may be cultivated and perfused with a similar solution which was used to remove the residual cord blood cells, preferably, without the addition of anticoagulant agents.

In certain embodiments of the invention, the drained, exsanguinated placenta is cultured as a bioreactor, i.e., an ex vivo system for propagating cells or producing biological materials. The number of propagated cells or level of biological material produced in the placental bioreactor is maintained in a continuous state of balanced growth by periodically or continuously removing a portion of a culture medium or perfusion fluid that is introduced into the placental bioreactor, and from which the propagated cells or the produced biological materials may be recovered. Fresh medium or perfusion fluid is introduced at the same rate or in the same amount.

The number and type of cells propagated may easily be monitored by measuring changes in morphology and cell surface markers using standard cell detection techniques such as flow cytometry, cell sorting, immunocytochemistry (e.g., staining with tissue specific or cell-marker specific antibodies) fluorescence activated cell sorting (FACS), magnetic activated cell sorting (MACS), by examination of the morphology of cells using light or confocal microscopy, or by measuring changes in gene expression using techniques well known in the art, such as PCR and gene expression profiling.

The growth factors introduced into the perfusion solution can stimulate the propagation of undifferentiated embryonic-like stem cells, committed progenitor cells, or differentiated cells (e.g., differentiated hematopoietic cells). The growth factors can stimulate the production of biological materials and bioactive molecules including, but not limited to, immunoglobulins, hormones, enzymes or growth factors as previously described. The cultured placenta should be “fed” periodically to remove the spent media, depopulate released cells, and add fresh media. The cultured placenta should be stored under sterile conditions to reduce the possibility of contamination, and maintained under intermittent and periodic pressurization to create conditions that maintain an adequate supply of nutrients to the cells of the placenta. It should be recognized that the perfusing and culturing of the placenta can be both automated and computerized for efficiency and increased capacity.

In another embodiment, the placenta is processed to remove all endogenous proliferating cells, such as embryonic-like stem cells, and to allow foreign (i.e., exogenous) cells to be introduced and propagated in the environment of the perfused placenta. The invention contemplates a large variety of stem or progenitor cells that can be cultured in he placental bioreactor, including, but not limited to, embryonic-like stem cells, mesenchymal stem cells, stromal cells, endothelial cells, hepatocytes, keratinocytes, and stem or progenitor cells for a particular cell type, tissue or organ, including but not limited to neurons, myelin, muscle, blood, bone marrow, skin, heart, connective tissue, lung, kidney, liver, and pancreas (e.g., pancreatic islet cells).

5.5 Methods of Treatment Using Assay-Identified Compounds

As shown in the working Examples (see Section 6, below), the assay identified a class of compounds that exhibit anti-angiogenesis activity. These compounds are representative members of the class of compounds described in Section 5.2, above. Specifically, the representative compounds are Actimid™, Revimid™ and thalidomide. Other compounds may be identified by the assay in the same manner as described in the Examples, and elsewhere herein. Such compounds may be any compound that has the desired modulatory effect on angiogenesis or vasogenesis, and may be a protein, peptide, peptide analog, nucleic acid or nucleic acid analog, carbohydrate, lipid, small inorganic molecule, etc.

Compounds identified as anti-angiogenic may be used to treat any disease or condition that has an angiogenic component. For example, one marker of aggressiveness in cancer, such as breast cancer, is the cancer tumor's production of angiogenic agents; and increase in vascularization within and peripheral to the tumor leads to an increased rate of tumor growth and chances for metastasis. Suppressing this angiogenic potential will help suppress growth and metastasis of the tumor. Thus, the anti-angiogenic compounds of the invention may be used to treat cancer, including metastatic cancer. Such treatment is preferably combined with other cancer therapies. Other disorders which may be treated with the compounds identified by the screening methods of the invention include inflammation, endometriosis, arthritis, atherosclerotic plaques, diabetic retinopathy, neovascular glaucoma, trachoma, corneal graft neovascularization, psoriasis, scleroderma, hemangioma and hypertrophic scarring, vascular adhesions and angiofibroma.

Thus, in one embodiment, the invention provides a method of treating an individual, wherein said individual has a condition or disease associated with angiogenesis or vasogenesis, comprising administering to said individual an amount of an agent sufficient to detectably reduce said angiogenesis or vasogenesis, wherein said agent has been identified in an assay described herein as having anti-angiogenic or anti-vasogenic activity. In a specific embodiment, said agent is a compound that suppresses the activity of TNF-α. In a more specific embodiment, said agent is selected from the group consisting of thalidomide, Actimid™ or Revimid™. In another embodiment, the invention provides a method of treating an individual, wherein said individual has a condition or disease associated with angiogenesis or vasogenesis, comprising administering to said individual an amount of a compound that suppresses the activity of TNF-α, wherein said amount is sufficient to detectably reduce said angiogenesis or vasogenesis. In a more specific embodiment, said compound is selected from the group consisting of thalidomide, Actimid™ or Revimid™.

The same method of identification may be used to identify compounds that increase vasogenesis or angiogenesis, i.e., angiogenic compounds; such agents may be used to treat diseases or conditions associated with insufficient vascularization, or an injury to vessels. For example, such compounds may be administered to individuals having undergone surgery, particularly vessel or cardiac surgery, to improve the rate of vessel repair. In a second example, such compounds may be used to treat individuals having insufficient peripheral blood flow, such as individual having a non-healing wound, or Reynaud's disease. Thus, in another embodiment, the invention provides a method of treating an individual, wherein said individual has a condition or disease associated with insufficient angiogenesis or vasogenesis, comprising administering to said individual an amount of an agent that detectably increases angiogenesis or vasogenesis, said agent administered in an amount sufficient to increase said angiogenesis or vasogenesis.

Modulators of angiogenesis and/or vasogenesis may be administered by the methods outlined in Section 5.6, below.

5.6 Pharmaceutical Compositions

The present invention encompasses pharmaceutical compositions comprising compounds identified to be modulators of angiogenesis by the methods of the present invention. The pharmaceutical compositions of the invention may be administered to a subject in need of such treatment in order to modulate angiogenesis.

Administration of compounds of the invention can be systemic or local. In most instances, administration to a mammal will result in systemic release of the compounds of the invention (i.e., into the bloodstream). Methods of administration include enteral routes, such as oral, buccal, sublingual, and rectal; topical administration, such as transdermal and intradermal; and parenteral administration. Suitable parenteral routes include injection via hypodermic needle or catheter, for example, intravenous, intramuscular, subcutaneous, intradermal, intraperitoneal, intraarterial, intraventricular, intrathecal, and intracameral injection and non-injection routes, such as intravaginal rectal, or nasal administration. Preferably, the compounds and compositions of the invention are administered orally. In specific embodiments, it may be desirable to administer one or more compounds of the invention locally to the area in need of treatment. This may be achieved, for example, by local infusion during surgery, topical application, e.g., in conjunction with a wound dressing after surgery, by injection, by means of a catheter, by means of a suppository, or by means of an implant, said implant being of a porous, non-porous, or gelatinous material, including membranes, such as sialastic membranes, or fibers.

The compounds of the invention can be administered via typical as well as non-standard delivery systems, e.g., encapsulation in liposomes, microparticles, microcapsules, capsules, etc. For example, the compounds and compositions of the invention can be delivered in a vesicle, in particular a liposome (see Langer, 1990, Science 249: 1527-1533; Treat et al., in Liposomes in Therapy of Infectious Disease and Cancer, Lopez-Berestein and Fidler (eds.), Liss, New York, pp. 353-365 (1989); Lopez-Berestein, ibid., pp. 317-327; see generally ibid.). In another example, the compounds and compositions of the invention can be delivered in a controlled release system. In one embodiment, a pump may be used (see Langer, supra; Sefton, 1987, CRC Crit. Ref. Biomed. Eng. 14: 201; Buchwald et al., 1980, Surgery 88: 507 Saudek et al., 1989, N. Engl. J. Med. 321: 574% In another example, polymeric materials can be used see Medical Applications of Controlled Release, Langer and Wise (eds.), CRC Press., Boca Raton, Fla. (1974); Controlled Drug Bioavailability, Drug Product Design and Performance, Smolen and Ball (eds.), Wiley, New York (1984); Ranger and Peppas, 1983, J. Macromol. Sci. Rev. Macromol. Chem. 23: 61; see also Levy et al., 1985, Science 228: 190; During et al., 1989, Ann. Neurol. 25: 351; Howard et al., 1989, J. Neurosurg. 71: 105). In still another example, a controlled-release system can be placed in proximity of the target area to be treated, e.g., the liver, thus requiring only a fraction of the systemic dose (see, e.g., Goodson, in Medical Applications of Controlled Release, supra, vol. 2, pp. 115-138 (1984)). Other controlled-release systems discussed in the review by Langer, 1990, Science 249: 1527-1533) can be used. When administered as a composition, a compound of the invention will be formulated with a suitable amount of a pharmaceutically acceptable vehicle or carrier so as to provide the form for proper administration to the mammal. The term “pharmaceutically acceptable” means approved by a regulatory agency of the Federal or a state government or listed in the U.S. Pharmacopeia or other generally recognized pharmacopeia for use in mammals, and more particularly in humans. The term “vehicle” refers to a diluent, adjuvant, excipient, or carrier with which a compound of the invention is formulated for administration to a mammal. Such pharmaceutical vehicles can be liquids, such as water and oils, including those of petroleum, animal, vegetable or synthetic origin, such as peanut oil, soybean oil, mineral oil, sesame oil and the like. The pharmaceutical vehicles can be saline, gum acacia, gelatin, starch paste, talc, keratin, colloidal silica, urea, and the like. In addition, auxiliary, stabilizing, thickening, lubricating and coloring agents may be used. Preferably, when administered to a mammal, the compounds and compositions of the invention and pharmaceutically acceptable vehicles, excipients, or diluents are sterile. An aqueous medium is a preferred vehicle when the compound of the invention is administered intravenously, such as water, saline solutions, and aqueous dextrose and glycerol solutions.

The present compounds and compositions can take the form of capsules, tablets, pills, pellets, lozenges, powders, granules, syrups, elixirs, solutions, suspensions, emulsions, suppositories, or sustained-release formulations thereof, or any other form suitable for administration to a mammal. In a preferred embodiment, the compounds and compositions of the invention are formulated for administration in accordance with routine procedures as a pharmaceutical composition adapted for oral or intravenous administration to humans. In one embodiment, the pharmaceutically acceptable vehicle is a hard gelatin capsule. Examples of suitable pharmaceutical vehicles and methods for formulation thereof are described in Remington: The Science and Practice of Pharmacy, Alfonso R. Gennaro ed., Mack Publishing Co. Easton, Pa., 19th ed., 1995, Chapters 86, 87, 88, 91, and 92, incorporated herein by reference.

Compounds and compositions of the invention formulated for oral delivery are preferably in the form of capsules, tablets, pills, or any compressed pharmaceutical form. Where in tablet or pill form, the compounds and compositions may be coated to delay disintegration and absorption in the gastrointestinal tract thereby providing a sustained action over an extended period of time. Selectively permeable membranes surrounding an osmotically active driving compound are also suitable for orally administered compounds and compositions of the invention. In these later platforms, fluid from the environment surrounding the capsule is imbibed by the driving compound that swells to displace the agent or agent composition through an aperture. These delivery platforms can provide an essentially zero order delivery profile as opposed to the spiked profiles of immediate release formulations. A time delay material such as glycerol monostearate or glycerol stearate may also be used. Oral compositions can include standard vehicles, excipients, and diluents, such as magnesium stearate, sodium saccharine, cellulose, magnesium carbonate, lactose, dextrose, sucrose, sorbitol, mannitol, starch, gum acacia, calcium silicate, microcrystalline cellulose, polyvinylpyrrolidinone, water, syrup, and methyl cellulose, the formulations can additionally include lubricating agents, such as talc, magnesium stearate, mineral oil, wetting agents, emulsifying and suspending agents, preserving agents such as methyl- and propylhydroxybenzoates. Such vehicles are preferably of pharmaceutical grade. Orally administered compounds and compositions of the invention can optionally include one or more sweetening agents, such as fructose, aspartame or saccharin; one or more flavoring agents such as peppermint, oil of wintergreen, or cherry; or one or more coloring agents to provide a pharmaceutically palatable preparation.

A therapeutically effective dosage regimen for the treatment of a particular disorder or condition will depend on its nature and severity, and can be determined by standard clinical techniques according to the judgment of a medical practitioner. In addition, in vitro or in vivo assays can be used to help identify optimal dosages. Of course, the amount of a compound of the invention that constitutes a therapeutically effective dose also depends on the administration route. In general, suitable dosage ranges for oral administration are about 0.001 milligrams to about 20 milligrams of a compound of the invention per kilogram body weight per day, preferably, about 0.7 milligrams to about 6 milligrams, more preferably, about 1.5 milligrams to about 4.5 milligrams. In a preferred embodiment, a mammal, preferably, a human is orally administered about 0.01 mg to about 1000 mg of a compound of the invention per day, more preferably, about 0.1 mg to about 300 mg per day, or about 1 mg to about 250 mg in single or divided doses. The dosage amounts described herein refer to total amounts administered; that is, if more than one compound of the invention is administered, the preferred dosages correspond to the total amount of the compounds of the invention administered. Oral compositions preferably contain 10% to 95% of a compound of the invention by weight. Preferred unit oral-dosage forms include pills, tablets, and capsules, more preferably capsules. Typically such unit-dosage forms will contain about 0.01 mg, 0.1 mg, 1 mg, 5 mg, 10 mg, 15 mg, 20 mg, 50 mg, 100 mg, 250 mg, or 500 mg of a compound of the invention, preferably, from about 5 mg to about 200 mg of compound per unit dosage.

In another embodiment, the compounds and compositions of the invention can be administered parenterally (e.g., by intramuscular, intrathecal, intravenous, and intraarterial routes), preferably, intravenously. Typically, compounds and compositions of the invention for intravenous administration are solutions in sterile isotonic aqueous vehicles, such as water, saline, Ringer's solution, or dextrose solution. Where necessary, the compositions may also include a solubilizing agent. Compositions for intravenous administration may optionally include a local anesthetic such as lignocaine to ease pain at the site of the injection. For intravenous administration, the compounds and compositions of the invention can be supplied as a sterile, dry lyophilized powder or water-free concentrate in a hermetically sealed container, such as an ampule or sachette, the container indicating the quantity of active agent. Such a powder or concentrate is then diluted with an appropriate aqueous medium prior to intravenous administration. An ampule of sterile water, saline solution, or other appropriate aqueous medium can be provided with the powder or concentrate for dilution prior to administration. Or the compositions can be supplied in pre-mixed form, ready for administration. Where a compound or composition of the invention is to be administered by intravenous infusion, it can be dispensed, for example, with an infusion bottle containing sterile pharmaceutical-grade water, saline, or other suitable medium.

Rectal administration can be effected through the use of suppositories formulated from conventional carriers such as cocoa butter, modified vegetable oils, and other fatty bases. Suppositories can be formulated by well-known methods using well-known formulations, for example see Remington: The Science and Practice of Pharmacy, Alfonso R. Gennaro ed., Mack Publishing Co. Easton, Pa., 19th ed., 1995, pp. 1591-1597, incorporated herein by reference.

To formulate and administer topical dosage forms, well-known transdermal and intradermal delivery mediums such as lotions, creams, and ointments and transdermal delivery devices such as patches can be used (Ghosh, T. K.; Pfister, W. R.; Yum, S. L Transdermal and Topical Drug Delivery Systems, Interpharm Press, Inc. p. 249-297, incorporated herein by reference). For example, a reservoir type patch design can comprise a backing film coated with an adhesive, and a reservoir compartment comprising a compound or composition of the invention, that is separated from the skin by a semipermeable membrane (e.g., U.S. Pat. No. 4,615,699, incorporated herein by reference). The adhesive coated backing layer extends around the reservoir's boundaries to provide a concentric seal with the skin and hold the reservoir adjacent to the skin.

The invention also provides pharmaceutical packs or kits comprising one or more containers filled with one or more compounds of the invention. Optionally associated with such container(s) can be a notice in the form prescribed by a governmental agency regulating the manufacture, use or sale of pharmaceuticals or biological products, which notice reflects approval by the agency of manufacture, use or sale for human administration. In one embodiment, the kit contains more than one compound of the invention. In another embodiment, the kit comprises a compound of the invention and another biologically active agent.

The compounds of the invention are preferably assayed in vitro and in vivo, for the desired therapeutic or prophylactic activity, prior to use in humans. For example, in vitro assays can be used to determine whether administration of a specific compound of the invention or a combination of compounds of the invention is preferred. The compounds and compositions of the invention may also be demonstrated to be effective and safe using animal model systems. Other methods will be known to the skilled artisan and are within the scope of the invention.

### 6. EXAMPLES

### 6.1 Example 1

### Human Angiogenesis Assay Development

Spontaneous Vasogenesis (Tube Formation) From Pluripotent Placental Stem Cells

Human pluripotent stem cells were plated immediately upon isolation and adherent cells were selected from non-adherent populations after 24 hours. These adherent cells were cultivated in DMEM supplemented with 10% cord blood serum (CBS) and antibiotics. The time course profile of spontaneous vasogenesis, as characterized by assembly of microtubular structures, was determined and cell specimens are collected at various time points to assay for endothelial specific markers and synthetic products. Based on time course thus obtained, treatment dose and timetables were developed to screen candidate angiogenesis modulatory chemicals.

Preparation of Umbilical Cord Blood Vessel Rings

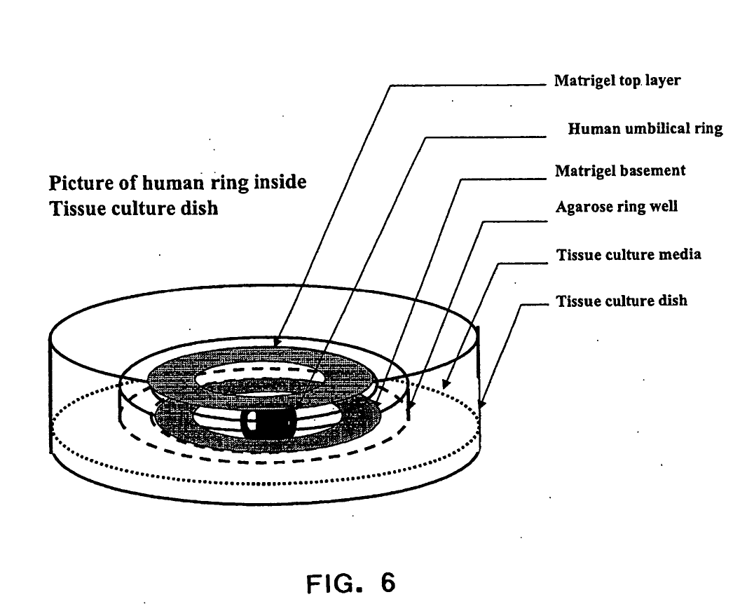

Blood vessels, approximately 1-2 mm in diameter and 1-2 cm in length, were excised from human umbilical cord within 12 hours of birth. Both arterial and venous tissue were harvested and maintained separately. The vessels were placed in DMEM containing 2.5 μg/ml of fungizone and cut into 1-2 mm length fragments using fine dissecting forceps and iridectomy scissors. Vessel fragments were freed of residual clots and soaked in DMEM before use. Dissecting and sectioning of vessels were performed with the aid of a surgical microscope. Similar angiogenic responses were obtained from blood vessels of venular and arterial origin but for each assay, vessel fragments from only one vessel were used. See FIG. 6 for a graphic depiction of the assay setu.

Assay Set-Up

Assays were performed in petri dishes (10 to 25 cm²) or 6-well culture plates (Costar, Cambridge, Mass.), which were prepared by pre-coating with either 0.1% gelatin (Sigma, St. Louis, Mo.) or MATRIGEL® (BD Biosciences) to form a matrix. Following the coating of the plates, 50 μl of human cord blood plasma in 5 mL of DMEM were added to each dish/well to form a surface film over the matrix. The film was allowed to set at 37° C. for 90 minutes, after which it was removed, leaving a thin film in each dish/well. Vessel ring segments were then positioned at central locations within the plate or dish. Petri dishes were divided into quarters, and vessel ring segments were placed in the center of each of the quadrants. In the case of 6-well culture plates, a vessel ring segment was placed in each of the wells. Vessel ring segments generally adhered to the coated matrix within 12 hours, allowing for the addition of media without risk of detachment due to buoyancy. Following adherence, vessels were cultured in DMEM supplemented with 20% human cord blood plasma, L-glutamine, penicillin/streptomycin and heparin, at 37° C. in a humidified environment for 14-21 days. The medium was changed at approximately 72 hour intervals.

Fibroblasts occasionally contaminated cultures, but usually only appeared as a monolayer on the bottom of the culture wells because, unlike endothelial cells, fibroblasts cannot invade MATRIGELs. Fibroblast outgrowth is negligible where vessel fragments are suspended in the fibrin gel rather than in contact with the plastic base of the culture wells. In order to inhibit clot retraction and resultant fibroblast contamination, the fibrinolytic inhibitor, epsilon-aminocaproic acid, was included in the culture medium.

Administration of Test Compounds and Scoring of Results

Test compounds were administered at the beginning of culture, once the adherent stem cells were selected, or once the vessel rings are determined to have adhered to the matrix. Each test compound is evaluated at various concentrations to enable generation of a dose response analysis.

Modulation of angiogenesis was defined as the change in angiogenesis in each assay as compared to a positive and a negative control. The positive control was defined as the response to endothelial cell growth supplement (ECGS; 200 μg/ml; Collaborative Research, Bedford, Mass.). The negative control was defined as the response to DMSO.

Vessel outgrowth was scored as a quantitative comparison to the positive and negative controls, using the following notations: − negative; +/− minimally above negative control; + low level of outgrowth; ++ moderate level of outgrowth; +++ high level of outgrowth; ++++ positive control level of outgrowth. Vessel outgrowth was also scored morphometrically as the maximal distance of vessel sprout growth in microns from the vessel ring, and as the total area of endothelial cell coverage (ECA)/area of vessel ring (VRA).

Migration Assay

Since vessel sprouts appear to be sensitive to the presence and nature of the extracellular matrix at the sprout origin, a modified method was used to simulate the physiologic extravascular environment. Using a non-denatured human collagen matrix (Anthromatrix), fixed human umbilical artery segments were cultured under the same conditions described above for vessel ring segments. As a modification, these vessel segments were mounted in a fixed position on the matrix. With this arrangement, endothelial sprouts grew onto the matrix, and an entire vessel segment “cassette” was recovered from the cell culture dish for analysis. The degree of angiogenesis was scored as above.

Immunohistochemistry

Plates demonstrating detectable angiogenic responses (i.e. new vascular growth) were fixed overnight in 4% paraformaldehyde in PBS at 4° C. in preparation for immunohistochemistry. The fixed matrices were paraffin-embedded. From these embedded matrices, 3 μm histological sections were cut and mounted on poly-L-lysine coated microscope slides. The sections were microwave-treated for 3 minutes and partially digested with 0.1% trypsin in 0.1% CaCl₂ in order to expose antigens. Sections were then reacted with antibodies and horseradish peroxidase-coupled sheep F(ab′)₂ anti-mouse Ig (Amersham, Amersham, Herts., U.K.) was used as the detection system. The sections were reacted with diaminobenzidine with silver enhancement and counterstained with haematoxylin. Antibodies used include monoclonal mouse anti-human factor VIII related antigen (Dako, Denmark), an anti-human endothelial cell mAb (Gibco, Grand Island, N.Y.) and a CD31-specific mAb (clone 20G5) produced in the John Curtin School of Medical Research.

Immunohistochemical staining of angiogenic samples was performed to detect Factor VIII related antigen, a reaction that clearly demonstrates whether outgrowths are blood vessels. The vessels were also reacted with a mAb specific for human endothelial cells (Gibco) and with a mAb to CD31, an antigen only expressed on endothelial cells, platelets and some leukocytes. In some cases, examination of angiogenic samples under the electron microscope was also performed to detect cells with a classic endothelial morphology.

Validating Method and Assay

Following culture for 14-21 days, as described above, angiogenesis was quantified and compared with control cultures.

The following substances were tested to establish baseline values:
heparin (100 μg/ml)
low molecular weight heparin (100 μg/ml)
suramin (a potent inhibitor of vascular endothelial growth factor) (100 μg/ml & 10 μg/ml)
3-hydrocortisone (10⁻⁵ M)
3-hydrocortisone (10⁻⁵ M) and heparin (100 μg/ml)
polyclonal neutralizing antibodies for acidic fibroblast growth factor (aFGF)
polyclonal neutralizing antibodies for basic fibroblast growth factor (bFGF)
mixture of polyclonal neutralizing antibodies for aFGF and bFGF
polyclonal neutralizing antibodies for vascular endothelial growth factor (VEGF).
   Developing Validation Criteria

These studies were performed to demonstrate that the system is effective in assaying known angiogenesis modulators.

Heparin and low molecular weight heparin (100 μg/ml) alone usually do not inhibit angiogenesis. Folkman & Brem (1992) “Angiogenesis and inflammation,” In: INFLAMMATION, BASIC PRINCIPLES AND CLINICAL CORRELATES Gallin et al., eds., Raven Press, New York. These two molecules, however, exhibited a small but significant inhibition of angiogenesis in the assay shown. However, this inhibitory effect may not reproduced in other assays. In contrast, suramin at 100 μg/ml virtually totally inhibited angiogenesis whereas at 10 μg/ml the inhibitory activity of this compound is lost. Hydrocortisone alone, like heparin, usually has little or no anti-angiogenic activity (Folkman & Brem (1992)). It is known that hydrocortisone, at the relatively high concentration of 10⁻⁵ M, partially inhibited angiogenesis compared with the DMSO (0.5%) diluent control [CITATION]. Here, however, a combination of heparin and hydrocortisone almost completely inhibited the angiogenic response. Such a result has been shown in vivo where heparin synergizes with steroids to cause regression of growing capillaries (Folkman & Brem (1992)).

Positive Controls

The growth factors acidic fibroblast growth factor (AFGF) and basic fibroblast growth factor (bFGF) are among the most potent angiogenic factors known. More recently vascular, endothelial growth factor (VEGF) has been identified as an important angiogenic factor, particularly in embryogenesis and solid tumours. A list of potential positive controls is provided in Table 1.

Table 1. Naturally-Occurring Stimulators of Angiogenesis

Proteins

Acidic fibroblast growth factor (aFGF)
Angiogenin
Basic fibroblast growth factor (bFGF)
Epidermal growth factor
Granulocyte colony stimulating factor
Hepatocyte growth factor
Interleukin 8
Placental growth factor
Platelet-denied endothelial growth factor
Scatter factor
Transforming growth factor alpha
Tumor necrosis factor alpha
Vascular endothelial growth factor (VEGF)
   Small Molecules
Adenosine
1-Butyryl glycerol
Nicotinamide
Prostaglandins E1 and E2

It was found that, compared with control antibodies, polyclonal neutralising antibodies against bFGF and AFGF partially inhibited angiogenesis, the anti-bFGF antibody being the more inhibitory of the two. In contrast, neutralising antibodies against VEGF had no effect on the angiogenic response. These data reveal that bFGF and aFGF, but not VEGF, play an important role in the in vitro angiogenesis assay described.

In order to quantify positive and/or maximal response, cultures were serum starved in order to reduce spontaneous angiogenesis. This step involved maintaining cultures in medium containing 20% human serum for the first 24 hours and then culturing the samples in serum free medium for the next 13-20 days with medium being changed every 3-4 days. Separate aliquots of substances suspected of possessing angiogenesis enhancing activity are added to individual wells as described above.

Developing Dose-Response Data for Known Pro-Angiogenesis Factors

Different concentrations of the angiogenic growth factors bFGF, AFGF and VEGF were evaluated to determine their ability to enhance angiogenesis in serum-starved cultures. Standard dose-response analyses were performed. Although the assay may be performed using “serum starved” culture conditions, media containing minimal serum constituents for endothelial cell survival were used when testing for substances that enhance angiogenesis.

Negative Controls

Similar dose-response analyses were made with factors known to have documented anti-angiogenic effects.

### 6.2 Example 2

### Effects of Thalomid™, Actimid™ and Revimid™ on Proliferation and Differentiation of Embryonic-Like Stem Cells Derived from Placenta

The following experiments evaluated the effects of Thalomid™, Actimid™ and Revimid™ on the morphological differentiation of embryonic-like stem cells derived from placenta. The morphological differentiation of cultured embryonic-like stem cells was evaluated after fourteen days of culture in the presence of placental conditioned medium and with DMSO (control), EGCF, Thalomid™, Actimid™ or Revimid™. Cells were examined and scored for the presence of various cell markers, as well as scored for morphological appearance, such as total area occupied in the culture dish and the amount of branching and/or bifurcation exhibited.

6.2.1 Materials and Methods

Embryonic-like stem cells were isolated from placenta as described above in Section 5.4. The embryonic-like stem cells were cultured using the culture conditions described above.

The cells were scored for the expression of CD34 (a marker of early hematopoietic progenitor cells; also an endothelial cell marker), CD45 (a marker of all hematopoietic cells except erythrocytes), CD105 (a marker of proliferating endothelial cells), smooth muscle cell (SMC)-specific myosin heavy chain, nestin (a marker of angiogenesis), and glial fibrillary acidic protein (GFAP). Ratios of CD34 cells/TNC (Total Number of Cells), CD45 cells/TNC and CD105 cells/TNC were also determined. Cells were also scored using inspection by light microscopy for total vessel area or field occupied, and for whether they exhibited branches or bifurcations.

6.2.2 Results and Discussion

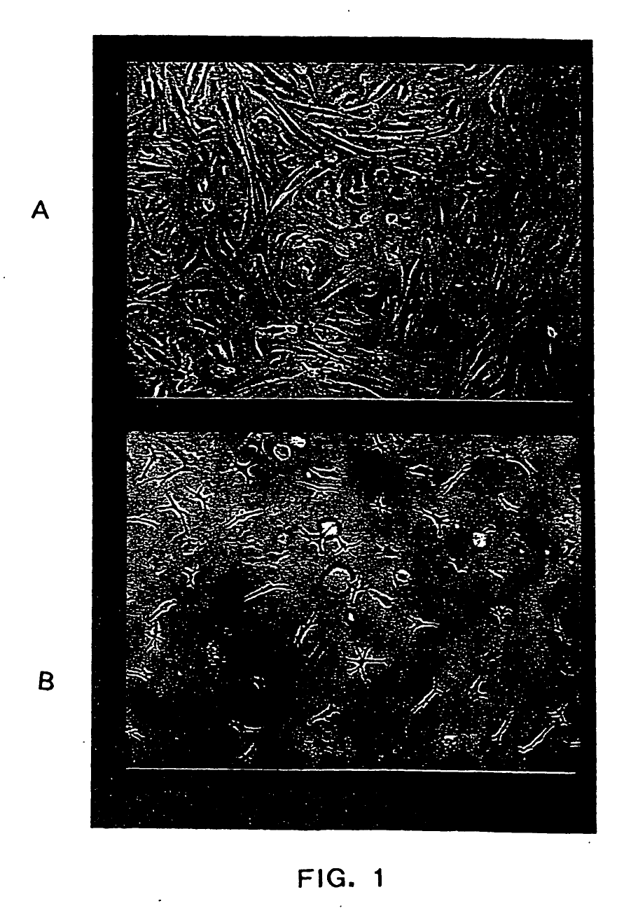

Tables 2-4 below, and FIGS. 1A-1C, summarize the results. In Table 2, the scoring was as follows:
−: no staining; +/−: <20% staining+: 20-50% staining; ++: 50-75% staining; +++: >75% staining.

The results in Table 2 show that numbers of cells expressing CD34, CD35 and smooth muscle cell (SMC)-specific myosin heavy chain decreased when cultured in the presence of Thalomid™, Actimid™, or Revimid™ and numbers of cells expressing nestin and glial fibrillary acidic protein (GFAP) increased.

TABLE 2
Effect of DMSO, Thalomid ™, Actimid ™ or Revimid ™ on the
Expression of CD34, CD45, Myosin Heavy chain, Nestin or GFAP

| | | | SMC- | | |
|---|---|---|---|---|---|
| | | | specific | | |
| | | | Myosin | | |
| Treatment Group | CD34+ | CD45+ | HC | Nestin | GFAP |
| Placental Conditioned | + | ++ | + | − | − |
| Media/DMSO | | | | | |
| Placental Conditioned | +/− | + | − | + | − |
| Media + Thalomid ™ | | | | | |
| 10 μg/mL | | | | | |
| Placental Conditioned | − | + | − | + | + |
| Media + Thalomid ™ | | | | | |
| 100 μg/mL | | | | | |
| Placental Conditioned | − | − | − | ++ | − |
| Media + Actimid ™ | | | | | |
| 1 μg/mL | | | | | |
| Placental Conditioned | − | − | − | +++ | + |
| Media + Actimid ™ | | | | | |
| 10 μg/mL | | | | | |
| Placental Conditioned | − | − | − | + | + |
| Media_Revimid ™ | | | | | |
| 1 μg/mL | | | | | |
| Placental Conditioned | − | − | − | ++ | +++ |
| Media + Revimid ™ | | | | | |
| 10 μg/mL | | | | | |

In another experiment, the results of which are summarized in Table 3, embryonic-like stem cells derived from placenta were cultured, using the conditions described in the umbilical vessel ring assay described above, in the presence of placenta-conditioned medium with DMSO (negative control), Thalomid™, Actimid™ or Revimid™. After 14 days in culture, the cells were then immunostained for expression of CD34+, CD45+ and CD 105+.

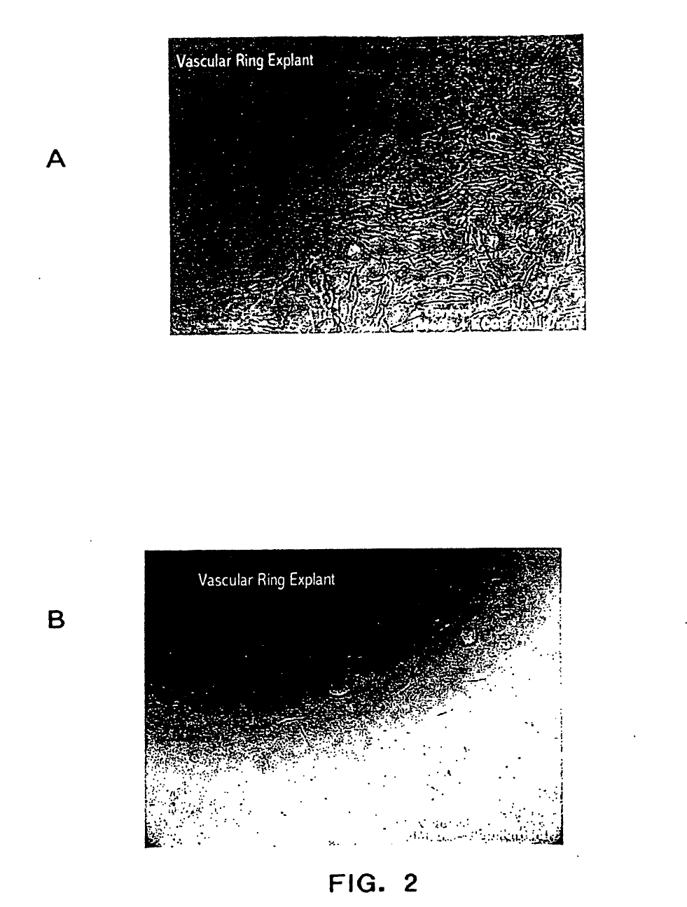

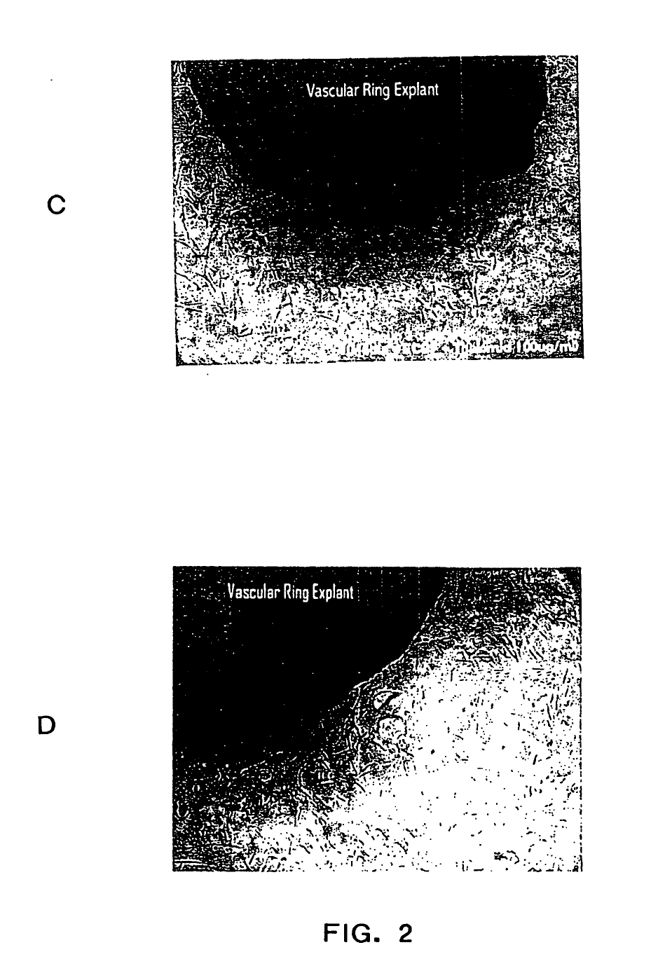

The results show that culturing in the presence of Thalomid™, Actimid™ or Revimid™ produces a decrease in the numbers of cells expressing CD34, CD45 and CD 105. See FIGS. 2A-2C.

TABLE 3
Effect of DMSO, Thalomid ™, Actimid ™ or Revimid ™
on the Expression of CD34, CD45 and CD 105 in Cultured Placental Stem
Cells

| | | | |
|---|---|---|---|
| | CD105/ | | |
| Treatment Group | TNC | CD34/TNC | CD45/TNC |

| | | | |
|---|---|---|---|
| Placental Conditioned | 33.7 | 2.37 | 9.44 |
| Media/DMSO | | | |
| Placental Conditioned Media + | 7.0 | 0.38 | 4.21 |
| Thalomid 100 μg/ml | | | |
| Placental Conditioned Media + | 6.8 | 0.64 | 3.87 |
| Thalomid 10 μg/ml | | | |
| Placental Conditioned Media + | 26.3 | 0.04 | 0.94 |
| Actimid ™ 1 μg/ml | | | |
| Placental Conditioned Media + | 17.3 | .22 | 1.71 |
| Revimid ™ 1 μg/ml | | | |

In another experiment, the results of which are summarized in Table 4, embryonic-like stem cells derived from placenta were cultured, using the culture conditions described above, and in the presence of EGCF, DMSO, Thalomid™, Actimid™ or Revimid™.

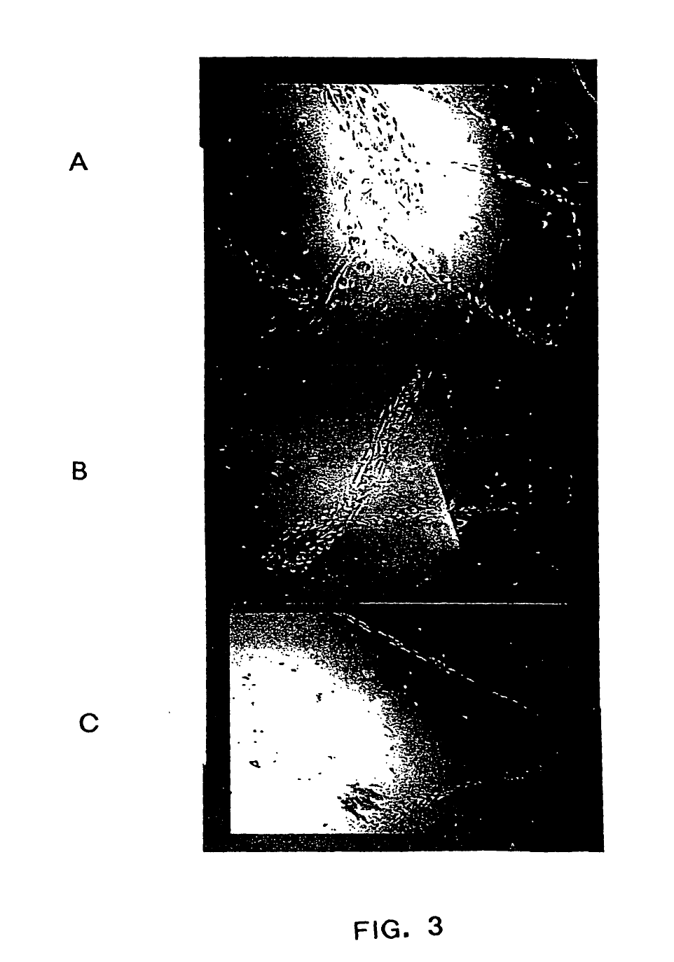

A “+” means that a branch or bifurcation was observed and a “−” means that no branch or bifurcation was observed. The results presented in Table 4 show that culturing placental embryonic-like stem cells in the presence of Thalomid™, Actimid™ or Revimid™ causes a decrease in the total vessel area/field covered by the cells, and also decreases the branching and/or bifurcation exhibited by the cells. See also FIGS. 3A, 3B

TABLE 4
Effect of ECGF, ECGF + DMSO, Thalomid ™, Actimid ™ or
Revimid ™ on Angiogenesis

| | Total Vessel | |
|---|---|---|
| | Area/Field | Branching/Bifurcation |
| Treatment Group | (% Coverage) | (+/−) |
| ECGF | 37.5 +/− 6.2 | + |
| ECGF + DMSO | 32.9 +/− 7.0 | + |
| 1 μg/ml | | |
| Thalomid ™ 1 μg/ml | 24.1 +/− 4.4 | − |
| Thalomid ™ 10 μg/ml | 14.8 +/− 7.2 | − |
| Actimid ™ 1 μg/ml | 11.3 +/− 2.8 | − |
| Actimid ™ 10 μg/ml | 6.7 +/− 4.1 | − |
| Revimid ™ 1 μg/ml | 13.5 +/− 7.7 | − |
| Revimid ™ 10 μg/mi | 12.1 +/− 7.4 | − |

### 6.3 Example 3

### Effects of Thalidomide in In Vitro Angiogenesis Assays

The following example demonstrates the effectiveness of the in vitro assays of invention to identify modulators of human angiogenesis. When compared to the in vitro assays of the prior art, e.g., rat aortic angiogenesis assay, the in vitro assays of the present invention demonstrate a higher level of specificity and sensitivity allowing for the detection of modulators of angiogenesis that would not be detected by prior art assays.

6.3.1 Rat Aortic Angiogenesis Assay:

Twelve well tissue culture grade plates were covered with 250 μl of Matrigel and allowed to gel for 30-45 min at 370 C, 5% Co2. Thoracic aortas were excised from eight to ten week old male Sprague Dawley rats and the fibroadipose tissue was removed. The aortas were sectioned into 1 mm long sections, rinsed eight times with EGM-2 (Clonetics Corp), placed on the Matrigel coated wells, covered with additional 250 μl Matrigel, and allowed to gel for 30-45 min at 370 C. The rings were cultured for 24 hours in 2 ml of EGM-2. After 24 hours, recombinant murine endostatin was reconstituted in EBM and added as a single treatment on day 1. Thalidomide was added at different concentrations (1 μg/ml, 5 μg/ml, 10 μg/ml, 50 μg/ml and 100 μg/ml) in the presence or absence of rabbit microsomes as noted in the Table 5. Aortic rings were photographed on days.

The results in the Table 5 indicated that thalidomide requires the addition of rabbit microsome in order to show efficient inhibition of vessel formation. Actimid™, however, did not require microsomes for inhibition of vessel formation.

TABLE 5
Effect of thalidomide on mean microvessel growth in the rat aortic
angiogenesis assay (expressed as & of control)

| | | | Thalidomide + | |
|---|---|---|---|---|
| | | Thalidomide | Rabbit | |
| | | (% of | microsomes | Actimid ™ |
| Concentrations | Control | control) | (% of control) | (% of control) |
| 10 μg/ml | 100 | 60 | 16.6 | 14.2 |
| 50 μg/ml | 100 | 82 | 17.6 | Not done |
| 100 μg/ml | 100 | Not done | Not done | 0.00 |

6.3.2 Human Angiogenesis

Fresh human umbilical cords were collected by trained medical personnel under fill donor informed consent from local hospitals. The cords were transported and treated within three hours. Umbilical cords and vessel lumens were rinsed with chilled basal nutrient medium. The artery was removed from the cord using mechanical means, forceps and small surgical scissors in an aseptic field. The vessel was cleaned of connective tissue and vessel rings were cut cross-wise in a length of 1 mm. The rings were placed into EGM-2 medium (Clonetics Corp.) in a 50 ml conical bottom tube and stored at 4° C. Six-well tissue culture plates were covered with 250 ml of Matrigel and allowed to gel for 30-45 min at 37° C., under 5% CO₂. The vessel rings were rinsed in EGM-2 medium and placed on the Matrigel-coated wells, covered with additional 250 μl Matrigel, and allowed to gel for 30-45 min at 37° C. (see FIG. 6). The vessels were cultured for 24 hours in 4 ml of EGM-2 to allow the tissue to adapt to its new environment. After 24 hours incubation, the rings were treated either with 0.1% DMSO as control, or different concentrations of compounds (thalidomide or CC4047). Culture medium was changed twice per week for total of three weeks.

The effects of compounds on cultured vessel rings were compared with the effect of DMSO on vessel rings. The results were analyzed using Image-Pro® Plus software (MediaCybernetics, Inc. Carlsbad, Calif.).

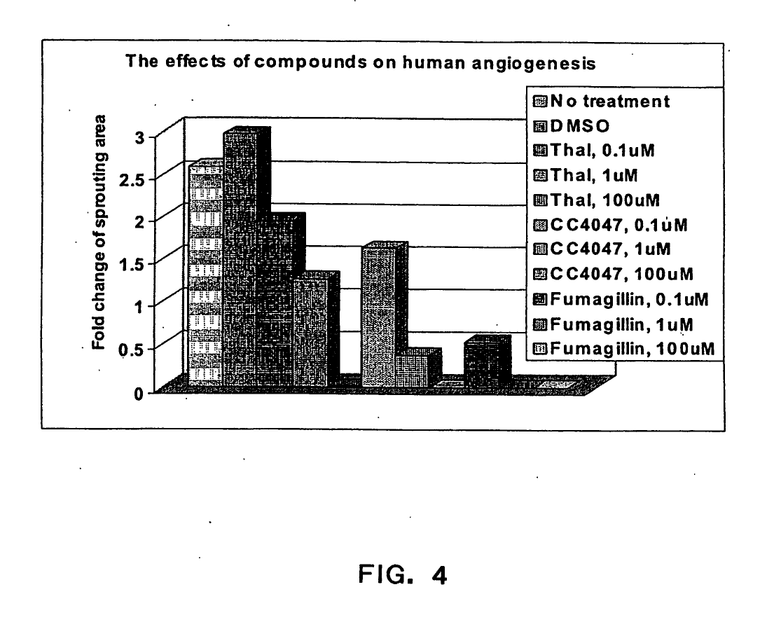

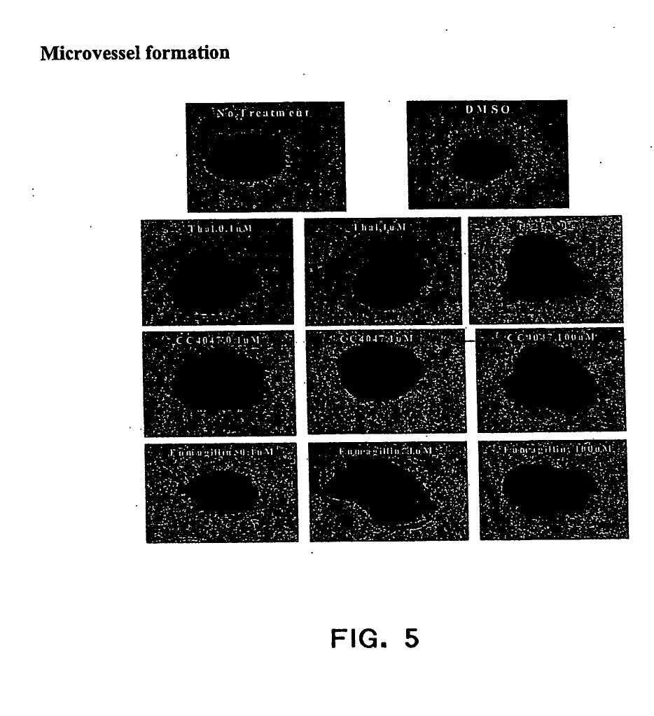

As is shown in Table 6 and FIGS. 4 and 5, both thalidomide and Actimid™ inhibited the formation of microvessel outgrowth in a dose dependent manner when they are compared with DMSO treated samples. These experiments were done in duplicates and the results are the average of two rings in same experiment. A different concentration of Fumagillin is used as positive control in this experiment.

TABLE 6
Effect of Thalidomide and Actimid on
Microvessel Growth in Human Angiogenesis Assay

| | | Thalidomide | Actimid ™ |
|---|---|---|---|
| Concentrations | | (% Inhibition) | (% Inhibition) |
| 0..1 | μM | 40 | 50.1 |
| 1 | μM | 81.4 | 85 |
| 10 | μM | 100 | 100 |

It is important to note that in this assay there is no need for either human or rabbit microsome for thalidomide to work (compare human ring results with rat ring results).

### 6.4 Example 4

### Assay for Angiogenesis Modulators Using Vessel Rings and Stem Cells

Vessel rings, at least ten, cultured individually, are co-cultured with stem cells to effectively re-create the vessel's natural environment. Vessel sections are obtained and plated as demonstrated in Example 1, above. Embryonic-like stem cells obtained from placenta are plated with the vessel sections, and both vessel section and stem cells are allowed to adhere. After 12 hours of culture, non-adherent stem cells are gently removed by washing. The cocultures are divided into at least two groups. One set of cocultures is then treated with DMSO as a control. The second set of cocultures is treated with a test compound. Other cocultures may be treated as positive controls, or other controls. The cocultures of stem cells and vessel sections are cultured for an additional 21 days. At the end of 21 days, control and test cocultures are examined and the extent of angiogenesis is determined by image scanning. Test cocultures demonstrate that the test compound is angiogenic where the average area of microvessel outgrowth is greater than the average area of vessel outgrowth for the control cocultures, and anti-angiogenic if the area is less than that of the control.

### 6.5 Example 5

### Assay for Angiogenesis Modulators Using Vessel Rings and Tumor Cells

Vessel rings, at least ten, cultured individually, are co-cultured with tumor cells to effectively re-create the vessel's natural environment within or peripheral to a tumor. Vessel sections are obtained and plated as demonstrated in Example 1, above. Tumor cells are obtained either from a tumor sample, or from a tumor cell line. Tumor cells are plated with the vessel sections to form cocultures, and both vessel section and stem cells are allowed to adhere. The cocultures are divided into at least two groups. One set of cocultures is treated with DMSO as a control. The second set of cocultures is treated with a test compound. Other cocultures may be treated as positive controls, or other controls. The cocultures of stem cells and vessel sections are cultured for an additional 21 days. At the end of 21 days, control and test cocultures are examined and the extent of angiogenesis is determined by image scanning. Test cocultures demonstrate that the test compound is angiogenic where the average area of microvessel outgrowth is greater than the average area of vessel outgrowth for the control cocultures, and anti-angiogenic if the area is less than that of the control.

The present invention is not to be limited in scope by the specific embodiments described herein. Indeed, various modifications of the invention in addition to those described herein will become apparent to those skilled in the art from the foregoing description. Such modifications are intended to fall within the scope of the appended claims.

### 7. REFERENCES

All references cited herein are incorporated herein by reference in their entirety and for all purposes to the same extent as if each individual publication, patent or patent application was specifically and individually indicated to be incorporated by reference in its entirety for all purposes.

The citation of any publication is for its disclosure prior to the filing date and should not be construed as an admission that the present invention is not entitled to antedate such publication by virtue of prior invention.
1. Folkman, J. and Brem, H. (1992) Angiogenesis and inflammation. In: “Inflammation, Basic Principles and Clinical Correlates”. Eds Gallin, J. I., Goldstein, I. M. and Snydennan, R. S., Raven Press, New York.
2. Folkman, J. (1985) Tumour angiogenesis. Adv. Cancer Res. 43, 175.
3. Folkman, J. and Klagsbrun, M. (1987). Angiogenic factors. Science 235, 442.
4. Folkman, J. (1985). Towards an understanding of angiogenesis: Search and discovery. Perspect. Biol. Med. 29, 10.
5. Langer, R. and Folkman, J. (1976). Polymers for the sustained release of proteins and other macromolecules. Nature 263. 797.
6. Montesano, R., Orci, L. and Vassalli, P. (1983). In vitro rapid organization of endothelial cells into capillary-like networks is promoted by collagen matrices. J. Cell. Biol. 97, 1648.
7. Madri, J. A. and Williams, S. K. (1983). Capillary endothelial cell cultures: phenotypic modulation by matrix components. J. Cell Biol. 97, 153.
8. Kubota, Y., Kleinnmann, H. K., Martin, G. R. and Lawley, T. (1988). Role of laminin and basement membrane in the morphological differentiation of human endothelial cells into capillary-like structures. J. Cell Biol. 107, 1589.
9. Leibovish, S. J., Polyerini, S. J., Shepard, H. M., Wiseman, D. M., Shively, V. and Nuseir, N. (1987). Macrophage-induced angiogenesis is mediated by tumor necrosis factor-alpha. Science 329, 640.
10. Montesano, R., Vassalli, J. D., Baird, A., Guillemin, R. and Orci, L. (1986). Basic fibroblast growth factor induces angiogenesis in vitro. Proc. Natl. Acad. Sci. 83, 7297.
11. Montesano, R., Pepper, M. S., Vassalli, J. D., and Orci, L. (1987). Phorbol ester induces cultured endothelial cells to invade a fibrin matrix in presence of fibrinolytic inhibitors. J. Cell. Physiol. 132, 509.
12. Nicosia, R. F. and Ottinetti, A. (1990). Growth of microvessels in serum-free matrix culture of rat aorta. A quantitative assay of angiogenesis in vitro. Lab Invest. 63, 115.
13. Knox, P., Crooks S. Scaife. M. C. and Patel, S. (1987). Role of plasminogen, plasmin and plasminogen activators in the migration of fibroblasts into plasma clots. J. Cell Physiol. 132, 501.
14. LaRocca, R. V., Stein, C. A., Danesi, R., Jamis-Dow, C. A., Weiss, G. H. and Myer, C. E. (1990). Suramin in adrenal cancer: modulation of steroid hormone production, cytotoxicity in vitro, and clinical antitumour effect. J. Clin. Endocrinol. Metab. 71, 497.

1. A method of identifying a modulator of angiogensis comprising:
(a) culturing a plurality of stem cells in the presence of a test compound, for a time and under conditions suitable for the growth endothelial cells; and
(b) comparing the amount of microvessel outgrowth from said stem cells in the presence of said test compound as compared to a control amount of microvessel outgrowth,
wherein if said microvessel outgrowth is greater or less than said control level of microvessel outgrowth, the test compound is identified as a modulator of angiogenesis.

2. The method of claim 1, wherein said stem cells are cultured with a vessel section.

3. The method of claim 1, wherein said stem cells are cultured with a plurality of tumor cells.

4. The method of claim 3, wherein said tumor cells are cells of a tumor cell line.

5. The method of claim 1, wherein said stem cells are additionally cultured in the presence of hydrocortisone, epidermal growth factor, or bovine brain extract.

6. The method of claim 1, wherein said modulator of angiogenesis is identified as an anti-angiogenic agent.

7. The method of claim 1, wherein said modulator of angiogenesis is identified as an angiogenic agent.

8. The method of claim 1, wherein said culturing of a plurality of stem cells in the presence of a test compound is for at least seven days.

9. The method of claim 1, wherein said culturing of a plurality of stem cells in the presence of a test compound is for at least fourteen days.

10. The method of claim 1, wherein said stem cells are cultured on a matrix that comprises fibrin.

11. The method of claim 1, wherein said stem cells are cultured in a physiological gel that comprises fibrin.

12. The method of claim 1, wherein said stem cells are cultured in a physiological gel that comprises non-denatured collagen.

13. A method of identifying a modulator of angiogensis comprising:
(a) culturing a vessel section in the presence of a plurality of tumor cells and a test compound, for a time and under conditions suitable for the growth of endothelial cells and said tumor cells; and
(b) comparing the amount of microvessel outgrowth from said vessel section in the presence of said test compound as compared to a control amount of microvessel outgrowth,
wherein if said microvessel outgrowth is greater or less than said control level of microvessel outgrowth, the test compound is identified as a modulator of angiogenesis.

14. A method of treating an individual, said individual having a disease or condition that is associated with abnormal vessel growth, comprising administering to said individual a therapeutically effective amount of a TNF-α inhibitor.

15. The method of claim 14, wherein said TNF-α inhibitor is an IMiD™.

16. The method of claim 15, wherein said IMiD™ is Actimid™ or Revimid™.

17. The method of claim 14, wherein said disease or condition is cancer.

18. The method of claim 17, wherein said cancer is a metastatic cancer.

19. The method of claim 17, wherein said cancer is breast cancer.

20. The method of claim 14, wherein said disease or condition is selected from the group consisting of inflammation, endometriosis, arthritis, atherosclerotic plaques, diabetic retinopathy, neovascular glaucoma, trachoma, corneal graft neovascularization, psoriasis, scleroderma, hemangioma and hypertrophic scarring, vascular adhesions and angiofibroma.

21. A method of inhibiting angiogenesis, comprising contacting a plurality of cells, said plurality of cells being capable of forming a vessel, with an inhibitor of TNF-α.

22. The method of claim 21, wherein said inhibitor of TNF-α is Actimid™ or Revimid™.

23. The method of claim 21, wherein said plurality of cells is a plurality of cells within an individual.

24. The method of claim 21, wherein said plurality of cells is a plurality of cells in cell culture.
